(19) United States
(12) Patent Application Publication
(10) Pub. No.: US 2017/0216240 A1
(43) Pub. Date: Aug. 3, 2017

(54) METHODS FOR MODULATING IKS CHANNEL ACTIVITY

(71) Applicants: Fredrik ELINDER, Hasselby (SE); UNIVERSITY OF MIAMI, Miami, FL (US)

(72) Inventors: Hans Peter Larsson, Miami, FL (US); Sara Liin, Linkoping (SE); Fredrik Elinder, Hasselby (SE)

(21) Appl. No.: 15/501,561

(22) PCT Filed: Aug. 4, 2015
(86) PCT No.: PCT/US2015/043622

§ 371 Date: Feb. 3, 2017

Related U.S. Application Data
(60) Provisional application No. 62/032,739, filed on Aug. 4, 2014.

(51) Int. Cl. A61K 31/202; A61K 31/185; A61K 31/19; A61K 45/06

(52) U.S. Cl. CPC A61K 31/202; A61K 45/06; A61K 31/185; A61K 31/19

(57) ABSTRACT

Disclosed herein are methods of using polyunsaturated fatty acids and derivatives thereof (“PUFAs”) to modulate Iₖₛ channels to treat conditions associated with a disruption in Iₖₛ channel activity, such as cardiac arrhythmias. In particular, disclosed herein are negatively charged PUFAs having decreased pKₐ values, which can activate (i.e., open) I_{Ks} channels, and positively charged PUFAs that can inhibit (i.e., close) I_{KS} channels.

### CROSS-REFERENCE TO RELATED APPLICATION

The benefit under 35 U.S.C. §119(e) of U.S. Provisional Patent Application Ser. No. 62/032,739 filed Aug. 4, 2014, is hereby claimed, and the disclosure thereof is hereby incorporated herein by reference.

### BACKGROUND OF THE INVENTION

Field of the Invention

The present disclosure relates to methods of using analogs of polyunsaturated fatty acids (“PUFAs”) as I_{Ks} channel modulators, and to treat conditions associated with a disruption in I_{Ks} channel activity.

Description of Related Technology

The cardiac action potential is mainly generated by sodium and calcium channels, which depolarize cardiomyocytes, and potassium channels, which repolarize cardiomyocytes, thereby terminating the action potential (Nerbonne et al. Physiol Rev 85, 1205-1253 (2005)). One of the major repolarizing cardiac potassium channels is the I_{Ks} channel. The I_{Ks} channel is formed by four α subunits (Kv7.1, originally called KCNQ1 or KvLQT1) and two to four auxiliary β subunits (KCNE1, originally called minK) (Nerbonne; Nakajo et al., Proc Natl Acad Sci USA 107, 18862-18867 (2010)).

The Kv7.1 is a tetrameric voltage-gated K (Kv) channel with six transmembrane segments (i.e., S1-S6) per subunit (Börjesson et al., Cell Biochem Biophys 52, 149-174 (2008)). The pore domain (with the central ion-conducting pore) is formed from all four subunits of helices S5 and S6. S6 has been shown to function as the activation gate, shutting off the intracellular access to the pore for K⁺ ions in the closed state of the channel (Börjesson et al. Cell Biochem Biophys 52, 149-174 (2008); Liu et al. Neuron 19, 175-184 (1997); Jiang, Y. et al. Nature 417, 523-526 (2002)). Helices 51 to S4 of each subunit form a voltage-sensor domain (VSD), which sense and respond to changes in the membrane potential. The fourth transmembrane segment, S4, in each subunit has several positively-charged amino acid residues and has been shown to move in response to changes in the transmembrane voltage (Börjesson et al., Cell Biochem Biophys 52, 149-174 (2008); Larsson et al., Neuron 16, 387-397 (1996)). In response to membrane depolarization, the voltage sensor S4 segments moves out of the plane of the membrane, triggering channel opening.

Although four Kv7.1 subunits per se form a functional channel, Kv7.1 needs to co-assemble with the auxiliary β subunit, KCNE1, to recapitulate the voltage dependence and kinetics of the native cardiac I_{Ks} channel (Sanguinetti, M. C. et al. Nature 384, 80-83 (1996); Barhanin, J. et al. Nature 384, 78-80 (1996)). KCNE1 is a single transmembrane helix protein suggested to associate with Kv7.1 in the lipid cleft between adjacent VSDs, making contact with VSD transmembrane segments S1 and S4 and pore transmembrane segment S6 (Shamgar, L. et al. PLoS ONE 3, e1943 (2008); Chung, D. Y. et al. Proc Natl Acad Sci USA 106, 743-748 (2009); Xu, X. et al., J Gen Physiol 1131, 589-603 (2008)).

More than 300 mutations in the genes encoding Kv7.1 and KCNE1 have been identified in patients with cardiac arrhythmia (Nerbonne et al. Physiol Rev 85, 1205-1253 (2005)). Loss-of-function mutations of the I_{Ks} channel tend to prolong the QT interval (Long QT syndrome), leading to ventricular arrhythmias, ventricular fibrillation, and sudden death (Nerbonne et al. Physiol Rev 85, 1205-1253 (2005)). Gain-of-function mutations of the I_{Ks} channel tend to shorten the QT interval (Short QT syndrome), leading to atrial fibrillation and formation of blood clots (Nerbonne et al. Physiol Rev 85, 1205-1253 (2005)). About 50% of patients suffering from Long QT syndrome harbor loss-of-function mutations in either Kv7.1 or KCNE (Börjesson et al., J Gen Physiol 137, 563-577 (2011)).

Therefore, there is a need for pharmacological augmentation (in the case of Long QT syndrome) or inhibition (in the case of Short QT syndrome) of I_{Ks} channel activity to treat these forms of cardiac arrhythmias.

### SUMMARY OF THE INVENTION

The invention provides a method of modulating an I_{Ks} channel comprising contacting the I_{Ks} channel with a compound, A-T, in an amount effective to modulate the I_{Ks} channel, wherein:

A is D(CH₂)ₘNHC(O), D(CH₂)ₘOC(O), D(CH₂)ₘSC(O), NH₂, NHR¹, N(R¹)₂, or N(R¹)₃⁺;
D is CO₂H, SO₃H, or OSO₃H;
T is C₅-C₂₉ alkenyl having at least two double bonds;
each R¹ independently is C₁₋₃ alkyl; and
m is 1-6.

In some embodiments, A-T modulates the I_{Ks} channel under physiological conditions. In various embodiments, the contacting occurs in vivo.

T can be, e.g., C₉₋₂₁ alkenyl, C₁₁₋₂₁ alkenyl, C₁₃₋₂₁ alkenyl, C₁₅₋₂₁ alkenyl, or C₁₉₋₂₁ alkenyl. Further, T can have at least three double bonds, at least four double bonds, at least five double bonds, at least six double bonds, or two to ten double bonds. In some embodiments, at least one double bond has cis stereochemistry, or at least two double bonds have cis stereochemistry, or each of the double bounds have cis stereochemistry. In some cases, T can be a linear alkenyl. In some embodiments, T comprises the structure:

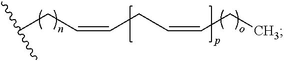

wherein n is 1-6; o is 0-6; and p is 1-7. For example, n can be 1 or 2 or 3 or 4 or 5 or 6. For example, o can be 0 or 1 or 2 or 3 or 4 or 5 or 6. For example, p can be 1 or 2 or 3 or 4 or 5 or 6 or 7.

In some cases, A-T activates the I_{Ks} channel. In these cases, A can be D(CH₂)ₘNHC(O), D(CHCH₃)ₘNHC(O), D(CH₂)ₘOC(O), or D(CH₂)ₘSC(O). For example, m can be 1 or 2 or 3 or 4 or 5 or 6. In embodiments wherein A is D(CH₂)ₘNHC(O), D can be COOH or SO₃H or OSO₃H. In embodiments wherein A is D(CH₂)ₘOC(O), D can be COOH or SO₃H or OSO₃H. In embodiments wherein A is D(CH₂)ₘSC(O), D can be COOH or SO₃H or OSO₃H. In cases where A-T activates the I_{Ks} channel, A-T can be selected from the group consisting of:

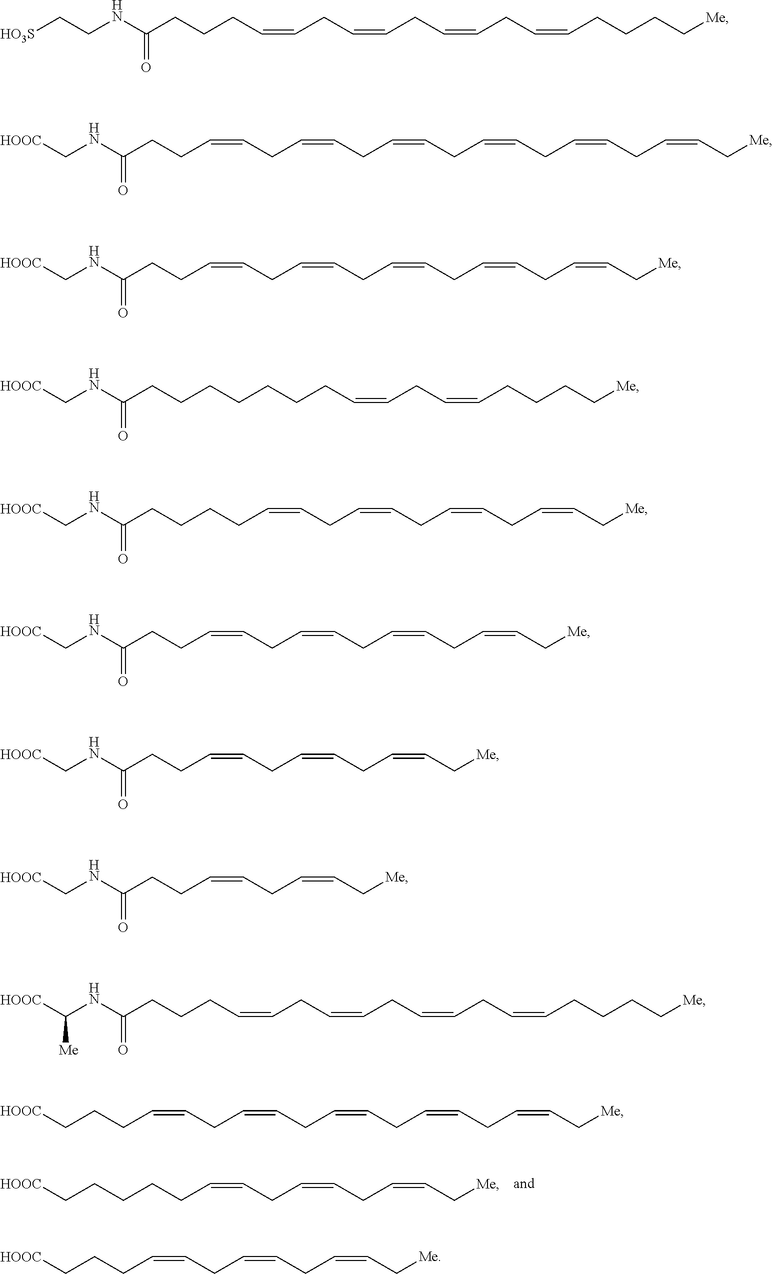

In these cases, the contacting can comprise administering A-T to a subject in need thereof. In some embodiments, the subject suffers from Long QT Syndrome, cardiac arrhythmia, atrial flutter, ventricular tachycardia, Romano-Ward syndrome, Jervell and Lange-Nielsen syndrome, or drug-induced Long QT syndrome.

In some cases, A-T inhibits the I_{Ks} channel. In these cases, A can be NH₂ or NHR¹ or N(R¹)₂ or N(R¹)₃. In embodiments wherein A is NHR¹, R¹ can be, e.g., CH₃. In embodiments wherein A is N(R¹)₂, each R¹ can be, e.g., CH₃. In embodiments wherein A is N(R¹)₃, each R¹ can be, e.g., CH₃. In cases wherein A-T inhibits the I_{Ks} channel, A-T can be:

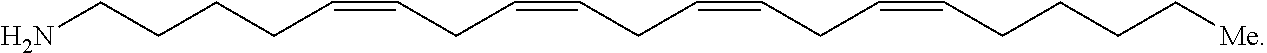 [SMILES: CCCCC/C=C\C/C=C\C/C=C\C/C=C\CCCCN]

In these cases, the contacting can comprise administering A-T to a subject in need thereof. In some embodiments, the subject suffers from Short QT Syndrome, blood clot formation, or atrial fibrillation.

Further aspects and advantages will be apparent to those of ordinary skill in the art from a review of the following detailed description, taken in conjunction with the drawings. While the PUFAs and methods disclosed herein are susceptible of embodiments in various forms, the description hereafter includes specific embodiments with the understanding that the disclosure is illustrative, and is not intended to limit the invention to the specific embodiments described herein.

### BRIEF DESCRIPTION OF THE DRAWINGS

FIG. 1A is a schematic illustration of PUFA modulation. PUFAs incorporate in the lipid cleft close to the channel's voltage-sensing domains (“VSD”) and electrostatically affect the S4 movement. +++ denotes S4 gating charges.

FIG. 1B is a schematic illustration of the effect of KCNE1-induced protonation of a negatively charged PUFA (upper) and a positively charged PUFA. ++++ denotes S4 gating charges.

FIG. 2 shows the effect of docosahexaenoic acid (“DHA”) on the voltage dependence of Kv7.1. (A) 70 μM DHA increases current amplitude of Kv7.1 in response to a −20 mV voltage step. (B) DHA shifts the G(V) of Kv7.1. DHA (), control (∘). The dashed curve in (B) is the control curve shifted −10 mV. (C) The concentration dependence of the DHA effect. Mean±SEM. ΔVₘₐₓ=−15.8 mV, c_{0.5}=50 μM. n=3-5. (D) DHA shifts the voltage dependence of Kv7.1 without altering the maximum conductance. Representative current families for the same cell as in FIG. 2A for control and 70 μM DHA on Kv7.1 channels. Currents for −20 mV are shown as bold traces.

FIG. 3 shows the effect of structure and pH on G(V) shifts. (A) Induced G(V) shifts for 70 μM of the indicated substance. Mean±SEM. n=3-8. (B) Schematic illustration of pH dependence of PUFA protonation. The probability of protonation of the PUFA is indicated by the gray scale of the (−): dark gray=low probability of being protonated, light gray=high probability of being protonated. (C) pH-dependence of the G(V) shift caused by the application of 70 μM DHA on Kv7.1. Mean±SEM. ΔVₘₐₓ=−30.7 mV, c_{0.5}=1.8*10⁻⁸=pH 7.7. n=3-5. (D) Concentration-response curves for DHA on Kv7.1 at different pHs. pH 9.0: ΔVₘₐₓ=−27.7 mV, C_{0.5}=10 μM; pH 7.4; n=3-8. (E & F) DHA methyl ester and DHA co-application reduces the DHA effect on Kv7.1. The G(V) shift induced by 70 μM DHA on a representative cell when DHA is applied alone (E) or together with 70 μM DHA methyl ester (F; DHA-me). DHA or DHA+DHA-me (), control (∘). The dashed line in (E) is the control curve shifted −11 mV and the dashed line in (F) is the control curve shifted −5.5 mV. (G) The same concentration-response curve as in FIG. 2C with experimental data for co-application added (arrow pointing to circle). Mean±SEM. n=4. The vertical arrow denotes the predicted G(V) shift by 35 μM DHA deduced from the concentration-response curve.

FIG. 4 shows that KCNE1 abolishes PUFA effect on Kv7.1/KCNE1 at physiological pH. (A-D) 70 μM DHA applied on Kv7.1/KCNE1 at pH 7.4 (A-B) and pH 9 (C-D). DHA (), control (∘). Bold traces=0 mV (A) or +10 mV (C). Dashed curve in (D) is control curve shifted −35 mV. (E-F) Kv7.1/KCNE1ΔC channel is insensitive to 70 μM DHA despite channel kinetics and voltage dependence being more similar to Kv7.1. DHA (), control (∘). Currents for −30 mV are shown as bold traces. (G-J) DHA effect is similar in 2:4 and 4:4 Kv7.1:KCNE1 concatemers. (G & H) 70 μM DHA induces a small shift in positive direction along the voltage axis for KCNE1-Kv7.1-Kv7.1 (forcing 4:2 Kv7.1:KCNE1 stoichiometry). DHA (), control (∘). Currents for 0 mV are shown as bold traces. The dashed line in (H) is the control curve shifted +7 mV. (I & J) 70 μM DHA induces a small shift in positive direction along the voltage axis also in KCNE1-Kv7.1 (forcing 4:4 Kv7.1:KCNE1 stoichiometry) channel voltage dependence. DHA (), control (∘). Currents for +10 mV are shown as bold traces. The dashed line in (J) is the control curve shifted +5.5 mV. (K) pH-dependence for 70 μM DHA. Kv7.1/KCNE1: =−26.6 mV, c_{0.5}=3.3*10⁻⁹=pH 8.5. n=3-4. For Kv7.1: see FIG. 3C.

FIG. 5 shows the effect of positively charged PUFAs on Kv7.1/KCNE1. (A) KCNE1 increases the effect of arachidonyl amine (AA+) on Kv7.1/KCNE1. (B) 70 μM AA+ at pH 7.4 induces a large shift in positive direction along the voltage axis in Kv7.1/KCNE1. AA+(), control (∘). Currents for +20 mV are shown as bold traces. The dashed line in (B) is the control curve shifted +28 mV. Note that the G(V) curves are from the same cell and not normalized. The continuous lines are Boltzmann curves (Eq. (1)) fitted to experimental data. The slope for AA+ is fixed to the same slope as for the control recording. (C) G(V) shift induced by 70 μM DHA or AA+. n=3-8.

FIG. 6 shows that PUFA analogues are effective on KV7.1/KCNE1. (A) 70 μM DHA-Glycine induces a negative G(V) shift of Kv7.1/KCNE1. DHA-Gly (D), control (∘). Dashed line is control curve shifted −20 mV. (B) DHA-Gly shifts the voltage dependence of activation of Kv7.1/KCNE.1. Representative current families for the same cell as in FIG. 6A for control and 70 μM DHA-Gly on Kv7.1/KCNE1 channels. Currents for −20 mV are shown as bold traces. (C) pH-dependence for 70 μM DHA-Gly or 70 μM DHA applied on Kv7.1/KCNE1. DHA-Gly: ΔVₘₐₓ=−31.6 mV, c_{0.5}=1.3*10⁻⁷=pH 6.9. n=3-5. For DHA: see FIG. 4K. (D) 70 μM N-arachidonoyl taurine (N-AT) induces a negative G(V) shift of Kv7.1/KCNE1. N-AT (), control (∘). Dashed line is control curve shifted −24 mV. (E) N-arachidonoyl taurine (N-AT) shifts the voltage dependence of activation of Kv7.1/KCNE1. Representative current families for the same cell as in FIG. 6D for control and 70 μMN-AT on Kv7.1/KCNE1 channels. Currents for 0 mV are shown as bold traces.

FIG. 7 shows that PUFAs target the VSD. (A) 70 μM DHA-Gly does not shift Kv7.1/R228Q. DHA-Gly (), control (∘). (B) shows that R228Q abolishes the effect of DHA-Gly on Kv7.1. Representative current families for control and 70 μM DHA-Gly on R228Q channels.

FIG. 8 shows that N-AT restores rhythmic firing in cardiomyocytes. (A-F) Representative examples of types of effects by application of 5 μM Chromanol 293B or 5 μM Chromanol 293B+30 μMN-AT on action potential frequency in isolated embryonic rat cardio mycocytes. (G-I) Effect of N-AT on action potential duration and frequency. Error bars denote SEM. n=5.

FIG. 9. shows the effect of N-AT on cardiomyocytes.

FIG. 10. shows that the PUFAs disclosed herein restore the QT interval in intact hearts subjected to drug-induced Long QT syndrome. (A) N-AT restores the QT interval in intact guinea pig hearts with prolonged QT induced by the drug E4031, a HERG channel blocker. (B) and (C) DHA-Gly restores the QT interval in intact guinea pig hearts with prolonged QT induced by E4031, as evidenced by the ECG (FIG. 10B) and ventricular surface action potential (FIG. 10C). (D) and (E) N-AT restores the QT interval in guinea pig hearts with prolonged QT induced by E4031, as evidenced by the ECG (FIG. 10D) and ventricular surface action potential (FIG. 10E).

FIG. 11. shows the effect of PUFA structure on Kv7.1 affinity. (A) shows the effect of different concentrations of linoleoyl-glycine (“Lin-Gly”) on Kv7.1 affinity. (B) shows the effect of different concentrations of stearidonyl-glycine (“Ste-Gly”) on Kv7.1 affinity. (C) compares the effect of NAA, Lin-Gly, Ste-Gly, DHA-Gly, TTA, and HTA on Kv7.1 affinity.

### DETAILED DESCRIPTION OF THE INVENTION

Provided herein are methods of modulating the I_{Ks} channel by contacting the channel with a polyunsaturated fatty acid having either a positive or a negative charge.

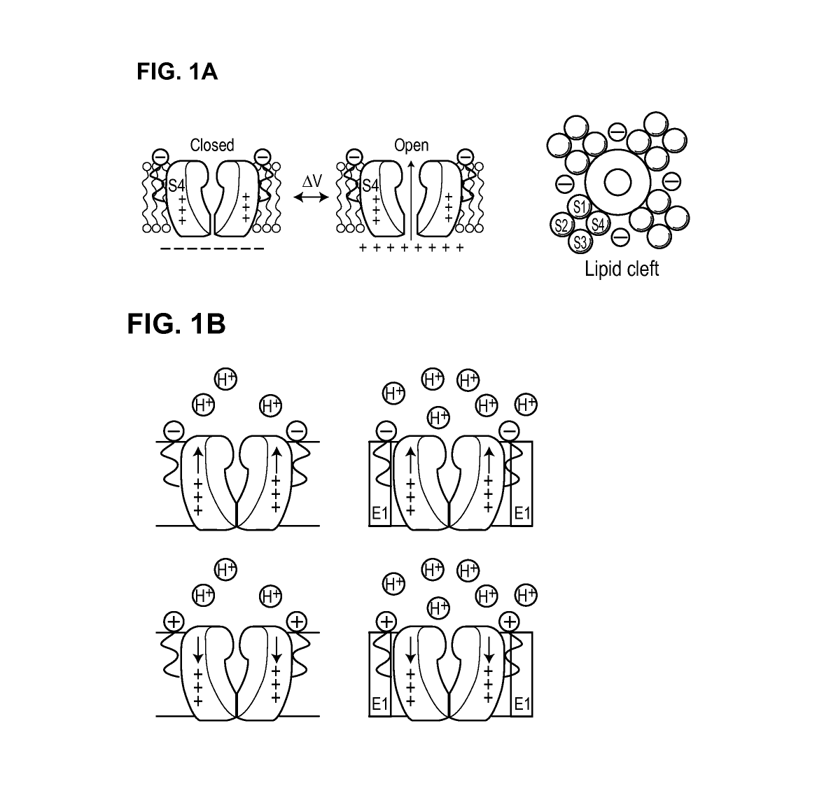

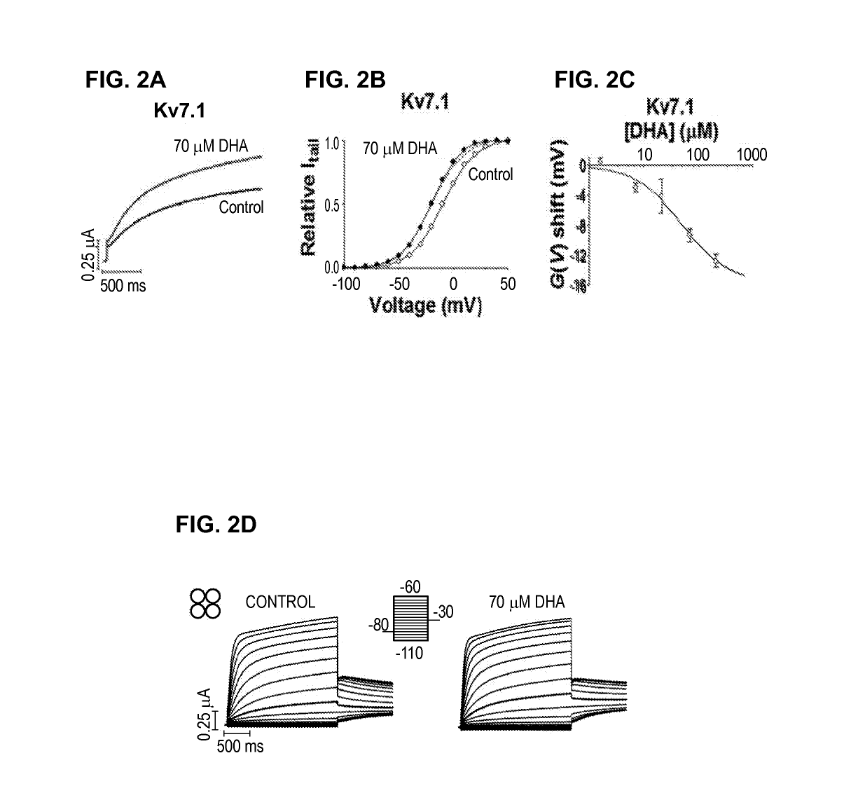

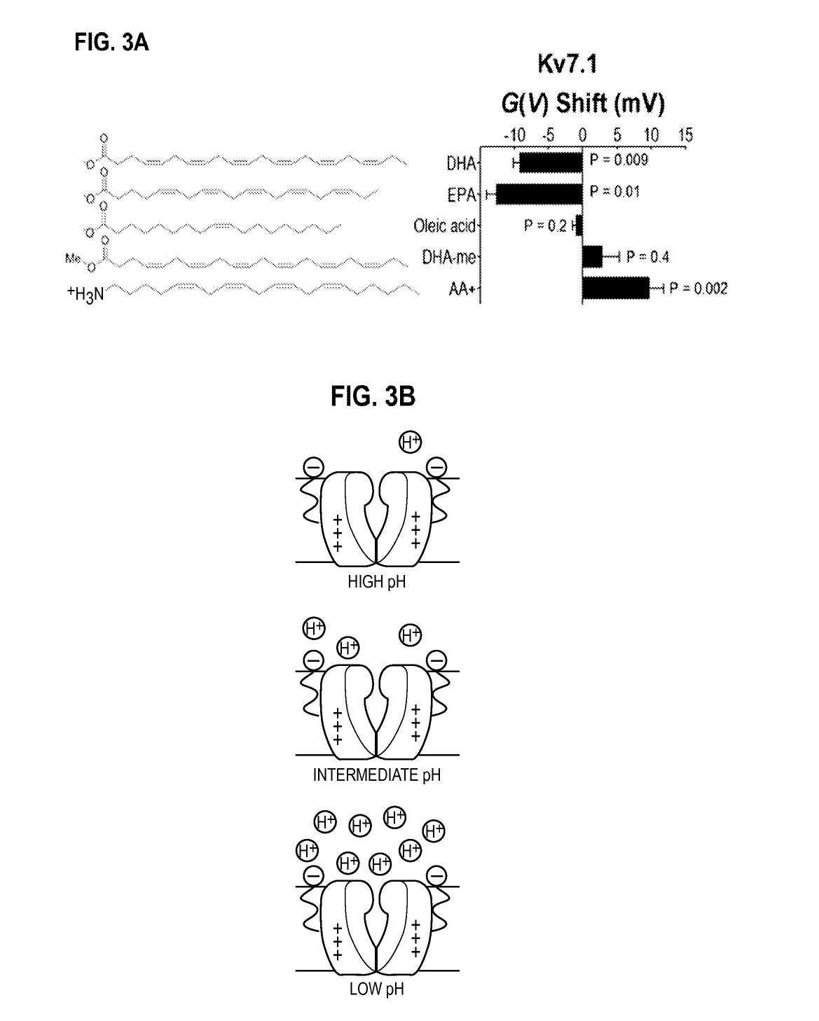

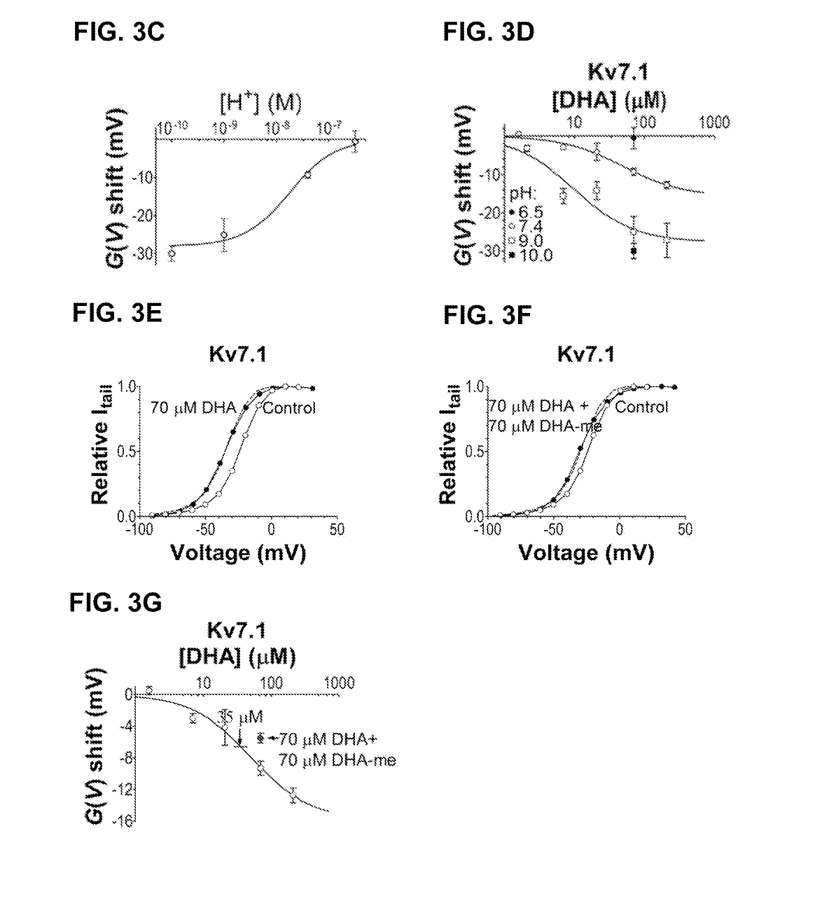

Polyunsaturated fatty acids (“PUFAs”) affect the Kv7.1 channel by an electrostatic effect on the voltage sensor S4. Negatively charged PUFAs open Kv7.1 by shifting the voltage dependence of Kv7.1 towards more negative membrane voltages. Without being bound by any particular theory, the PUFA polyunsaturated tail locates to the otherwise lipid filled space between two neighboring voltage-sensing domains (“VSDs”), and the negatively-charged group electrostatically interacts with the VSD to facilitate channel opening (illustrated in FIG. 1A). For example, docosahexaenoic acid (“DHA”) and eicosapentaenoic acid (“EPA”) each can shift the G(V) curve of the Kv7.1 channel in the negative direction along the voltage axis by about −9.3±0.9 mV and about −12.7±1.4 mV, respectively (Example 1, FIG. 2A-D, 3A, Table 1). Unsaturated and neutral fatty acids, however, have no effect on the G(V) curve. For example, docosahexaenoic acid methyl ester (“DHA-Me) and oleic acid (“OA”) shift the G(V) curve by +2.8±2.5 mV and −1.0±0.5 mV, respectively (Example 1, FIG. 3A, Table 1).

The ability of PUFAs to affect the Kv7.1 channels depends on the external pH of the binding site. PUFAs in a lipid environment have an apparent pKₐ of about 7 (Xiong et al. Nat Chem Biol 3, 287-296 (2007); Hamilton, J. A. J Lipid Res 39, 467-481 (1998)). As a result, about 50% of the PUFA molecules are deprotonated and negatively charged at pH 7.4, while about 50% are protonated and uncharged. Thus, increasing the external pH increases the shift in negative direction of the G(V) curve protocol, whereas reducing external pH to 6.5 abolishes the PUFA effect (Example 1, FIG. 3B-D).

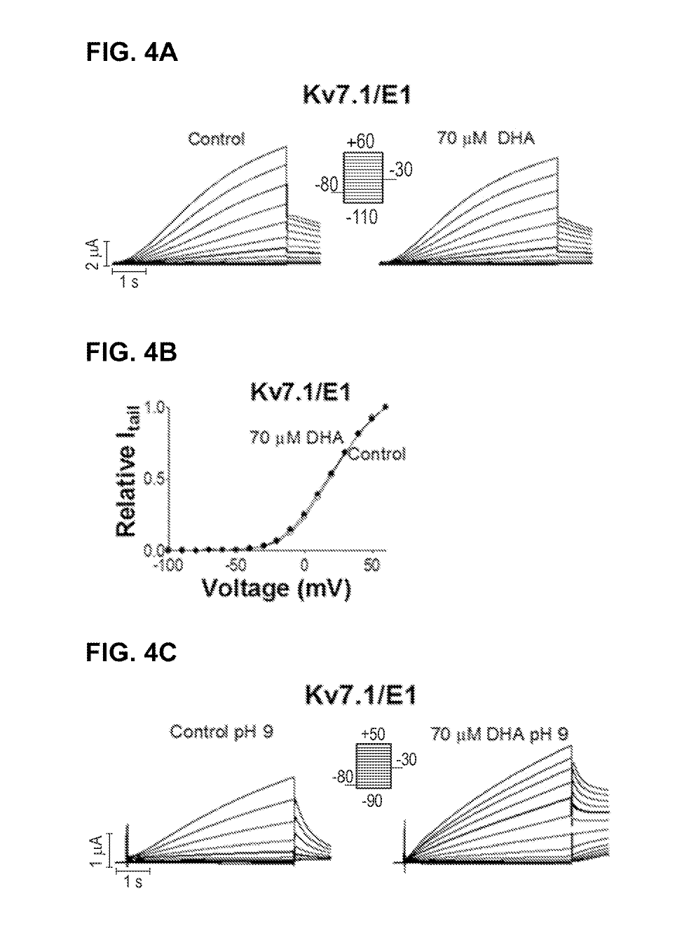

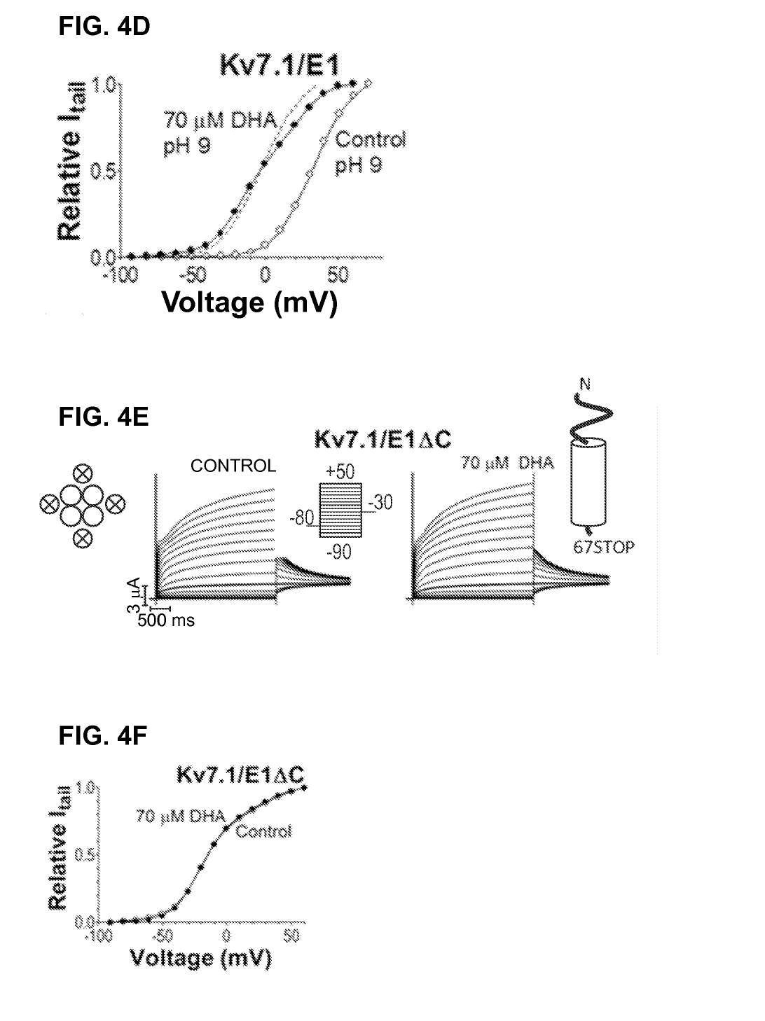

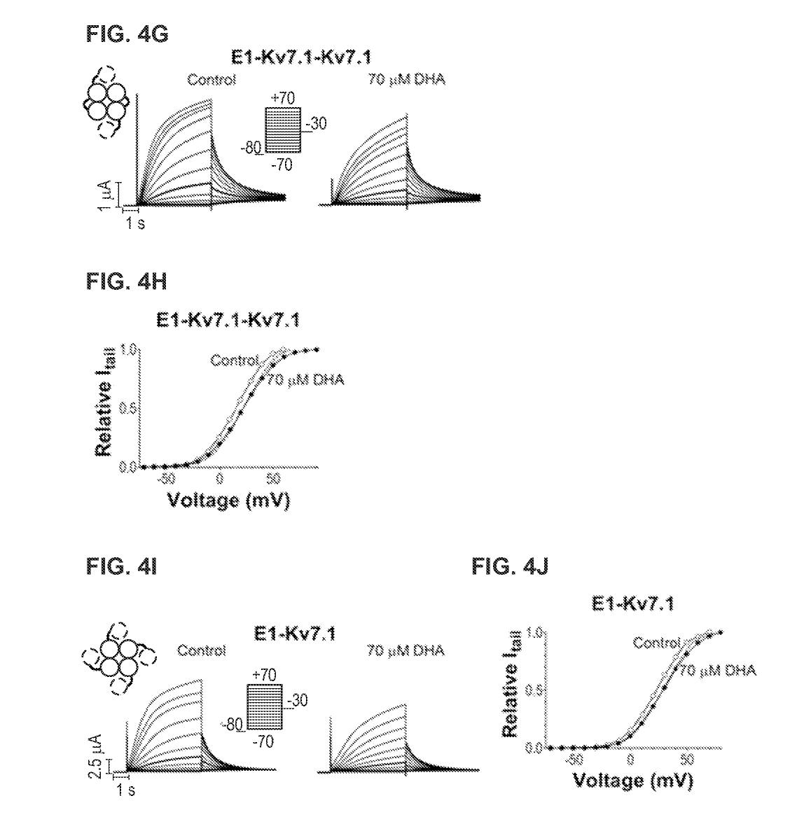

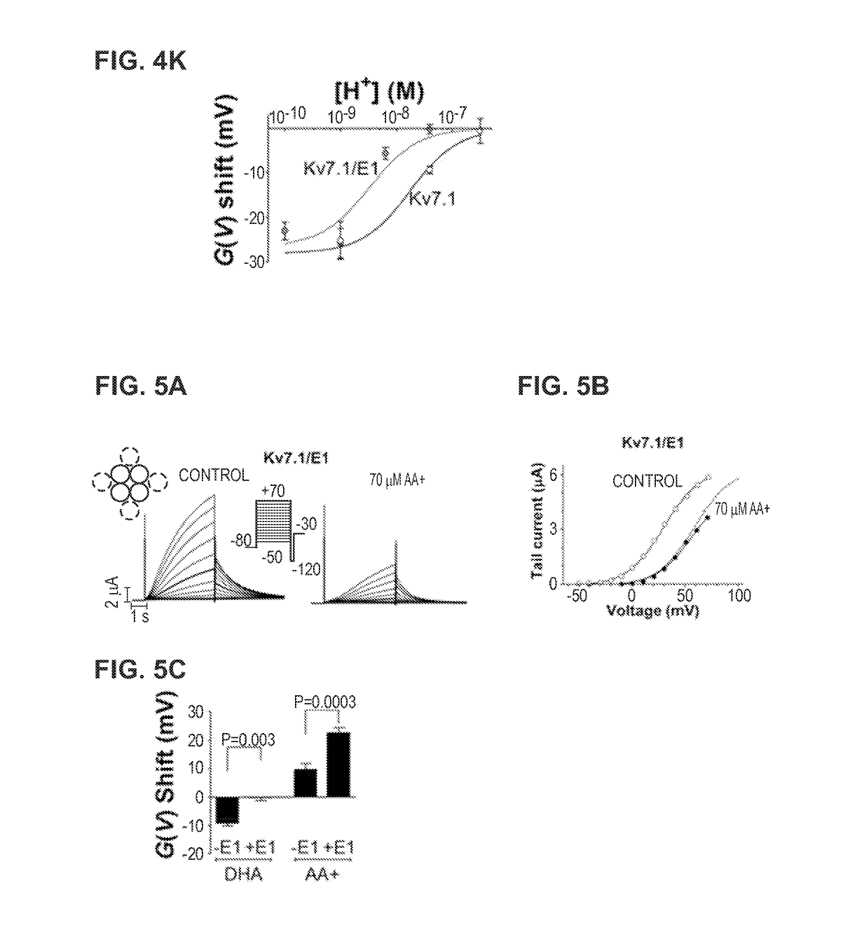

The auxiliary β subunit KCNE1 is necessary for Kv7.1 to form the cardiac I_{Ks} channel, but it has been found that KCNE1 co-expression decreases the effect of negatively charged PUFAs on Kv7.1 in a pH-dependent manner (FIG. 1B upper panel). Without being bound by any particular theory, the KCNE1 alters the local proton concentration in the PUFA binding site, decreasing the local pH. As a result, a negatively charged PUFA is protonated in the presence of KCNE1, which neutralizes the PUFA and eliminates the electrostatic effect of the PUFA on the channel. Therefore, traditional PUFAs do not act on I_{Ks} channels in vivo. For example, DHA could not induce a G(V) shift of Kv7.1 when Kv7.1 was co-expressed with KCNE1 at physiological pH (Example 2, FIG. 4A-B, E-F).

Furthermore, the presence of KCNE1 increases the Kv7.1 channel sensitivity to positively charged PUFAs by promoting amine protonation, thereby shifting the G(V) curve in the positive direction (FIG. 1B lower panel). Without being bound by any particular theory, the positive shift induced by the positively charged PUFA is caused by electrostatic repulsion of the positively charged amine group and the S4 voltage sensor.

Thus, disclosed herein are PUFAs having a general formula A-T, wherein A is a charged moiety and T is an alkenyl chain. These PUFAs can bypass the auxiliary subunit interference of KCNE1 to modulate activity of the I_{Ks} channel (e.g., at physiological pH). In particular, disclosed herein are negatively charged PUFAs having decreased pKₐ values, which can activate (i.e., open) I_{Ks} channels, and positively charged PUFAs that can inhibit i.e., close) I_{Ks} channels. Further disclosed herein are methods of using the PUFAs to modulate I_{Ks} channels and to prevent cardiac arrhythmias. Also disclosed herein are methods of treating or preventing drug-induced Long QT syndrome by administering the PUFAs along with a second therapeutic agent that can potentially induce Long QT syndrome, such as an anti-cancer drug.

The PUFAs described herein can shift the voltage dependence of the I_{Ks} channel (e.g., up to about ±50 mV). Therefore, they can restore the function of even the most severe LQTS and SQTS mutations. Further, it has been found that different LQTS and SQTS mutants can induce different shifts in the voltage dependence of I_{Ks} channels. Advantageously, the size of shift in voltage dependence in I_{Ks} channels depends on the pKₐ of the PUFA. Therefore, the PUFAs described herein can be tailored to restore wild type behavior in a various different types of LQTS and SQTS mutations.

### Definitions

As used herein, “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄ alkyl refers to an alkyl group that has 4 carbon atoms. C₁-C₇ alkyl refers to an alkyl group having a number of carbon atoms encompassing the entire range (i.e., 1 to 7 carbon atoms), as well as all subgroups (e.g., 1-6, 2-7, 1-5, 3-6, 1, 2, 3, 4, 5, 6, and 7 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

The term “alkenyl” is defined identically as “alkyl” except for containing at least one carbon-carbon double bond, and having two to thirty carbon atoms, for example, two to twenty carbon atoms, or two to ten carbon atoms. The term Cₙ means the alkenyl group has “n” carbon atoms. For example, C₄ alkenyl refers to an alkenyl group that has 4 carbon atoms. C₂-C₇ alkenyl refers to an alkenyl group having a number of carbon atoms encompassing the entire range (i.e., 2 to 7 carbon atoms), as well as all subgroups (e.g., 2-6, 2-5, 3-6, 2, 3, 4, 5, 6, and 7 carbon atoms). Unless otherwise indicated, an alkenyl group can be an unsubstituted alkenyl group or a substituted alkenyl group.

The term “alkynyl” is defined identically as “alkyl” except for containing at least one carbon-carbon triple bond, and having two to thirty carbon atoms, for example, two to twenty carbon atoms, or two to ten carbon atoms. The term G means the alkynyl group has “n” carbon atoms. For example, C₄ alkynyl refers to an alkynyl group that has 4 carbon atoms. C₂-C₇ alkynyl refers to an alkynyl group having a number of carbon atoms encompassing the entire range (i.e., 2 to 7 carbon atoms), as well as all subgroups (e.g., 2-6, 2-5, 3-6, 2, 3, 4, 5, 6, and 7 carbon atoms). Unless otherwise indicated, an alkynyl group can be an unsubstituted alkynyl group or a substituted alkynyl group.

As used herein, the term “therapeutically effective amount” means an amount of a compound or combination of therapeutically active compounds (e.g., a PUFA or combination of PUFAs) that ameliorates, attenuates or eliminates one or more symptoms of a particular disease or condition (e.g., LQTS or SQTS), or prevents or delays the onset of one of more symptoms of a particular disease or condition.

As used herein, the terms “patient” and “subject” may be used interchangeably and mean animals, such as dogs, cats, cows, horses, and sheep (i.e., non-human animals) and humans. Particular patients are mammals (e.g., humans). The term patient includes males and females.

As used herein, the term “pharmaceutically acceptable” means that the referenced substance, such as a compound of the present invention, or a formulation containing the compound, or a particular excipient, are safe and suitable for administration to a patient. The term “pharmaceutically acceptable carrier” refers to a medium that does not interfere with the effectiveness of the biological activity of the active ingredient(s) and is not toxic to the host to which it is administered.

As used herein the terms “treating”, “treat” or “treatment” and the like include preventative (e.g., prophylactic) and palliative treatment.

As used herein, the term “excipient” means any pharmaceutically acceptable additive, carrier, diluent, adjuvant, or other ingredient, other than the active pharmaceutical ingredient (API).

As used herein, the terms “KCNE1-Kv7.1” and “KCNE1-Kv7.1-Kv7.1” refer to concatemers of Kv7.1 and KCNE1 in 4:4 and 4:2 stoichiometry, respectively.

As used herein, the term “Kv7.1/KCNE1” refers to channels wherein Kv7.1 is co-expressed with KCNE1.

### Modulating I_{K} Channels

Disclosed herein are PUFAs that modulate the activity of the Kv7.1 channel, whether or not the Kv7.1 channel is co-expressed with KCNE1 (“Kv7.1/KCNE1”). The negatively charged PUFAs of the disclosure can shift the voltage dependence of the I_{Ks} channel in the negative direction, thereby activating the channel. The positively charged PUFAs of the disclosure can shift the voltage dependence of the I_{Ks} channel in the positive direction, thereby inhibiting the channel.

The degree of the shift in voltage depends on the pKₐ value of the negatively or positively charged PUFAs. The lower the pKₐ, the greater the degree of voltage dependence shift in the negative direction. The higher the pKₐ, the greater the degree of voltage dependence shift in the positive direction. For example, the negatively charged PUFAs disclosed herein can shift the voltage dependence of the I_{Ks} channel up to about −5 mV, or up to about −10 mV, or up to about −15 mV, or up to about −20 mV, or up to about −25 mV, or up to about −30 mV, or up to about −35 mV, or up to about −40 mV, or up to about −45 mV, or up to about −50 mV at physiological pH. In some cases, the negatively charged PUFAs disclosed herein can shift the voltage dependence of the I_{Ks} channel in a range of about −5 mV to about −50 mV, or about −5 mV to about −30 mV, or about −10 mV to about −40 mV, or about −15 mV to about −30 mV, or about −20 mV to about −30 mV, or about −30 mV to about −50 mV.

For example, the positively charged PUFAs disclosed herein can shift the voltage dependence of the I_{Ks} channel up to about +5 mV, or up to about +10 mV, or up to about +15 mV, or up to about +20 mV, or up to about +25 mV, or up to about +30 mV, or up to about +35 mV, or up to about +40 mV, or up to about +45 mV, or up to about +50 mV at physiological pH. In some cases, the positively charged PUFAs disclosed herein can shift the voltage dependence of the I_{Ks} channel in a range of about +5 mV to about +50 mV, or about +5 mV to about +30 mV, or about +10 mV to about +40 mV, or about +15 mV to about +30 mV, or about +20 mV to about +30 mV, or about +30 mV to about +50 mV.

The PUFAs of the disclosure can be represented by A-T, wherein A is a charged group and T is an alkenyl group.

### PUFA Tail (“T”)

The PUFA's of the disclosure include an alkenyl group (“T”).

T comprises C₅₋₂₉ alkenyl. In some embodiments, T can comprise C₉₋₂₁ alkenyl, or C₁₁₋₁₂ alkenyl, or C₁₃₋₂₁ alkenyl, or C₁₅₋₂₁ alkenyl, or C₁₉₋₂₁ alkenyl. For example, T can comprise C₅ or C₆ or C₇ or C₈ or C₉ or C₁₀ or C₁₁ or C₁₂ or C₁₃ or C₁₄ or C₁₅ or C₁₆ or C₁₇ or C₁₈ or C₁₉ or C₂₀ or C₂₁ or C₂₂ or C₂₃ or C₂₄ or C₂₅ or C₂₆ or C₂₇ or C₂₈ or C₂₉ alkenyl. Specifically contemplated T's include C₁₅ or C₁₆ or C₁₇ or C₁₈ or C₁₉ or C₂₀ or C₂₁ alkenyl (e.g., C₁₉ or C₂₀ or C₂₁ alkenyl).

The alkenyl group of T includes at least two double bonds. In some embodiments, T can include at least three double bonds, or at least four double bonds, or at least five double bonds, or at least six double bonds. In some cases, T can include two to ten double bonds, or two to six double bonds, or three to six double bonds.

At least one double bond of T has cis stereochemistry. In some embodiments, at least two double bonds of T have cis stereochemistry (e.g., at least two, or at least three, or at least four, or at least five, or at least six double bonds). In some cases, each double bond of T has cis stereochemistry.

T can be a linear or branched alkenyl. In some embodiments, T is a linear alkenyl. In other embodiments, T is a branched alkenyl.

T can further include at least one triple bond (e.g., 1, or 2, or 3, or 4, or 5 triple bonds).

In various cases, T comprises the structure:

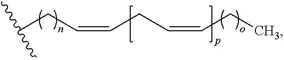

wherein n is 1-6 (e.g., 1 or 2 or 3 or 4 or 5 or 6), o is 0-6 (e.g., 0 or 1 or 2 or 3 or 4 or 5 or 6), and p is 1-7 (e.g., 1 or 2 or 3 or 4 or 5 or 6 or 7). For example, n can be 1-4, p can be 1-5, and o can be 1-4. In some cases, T is selected from, for example:

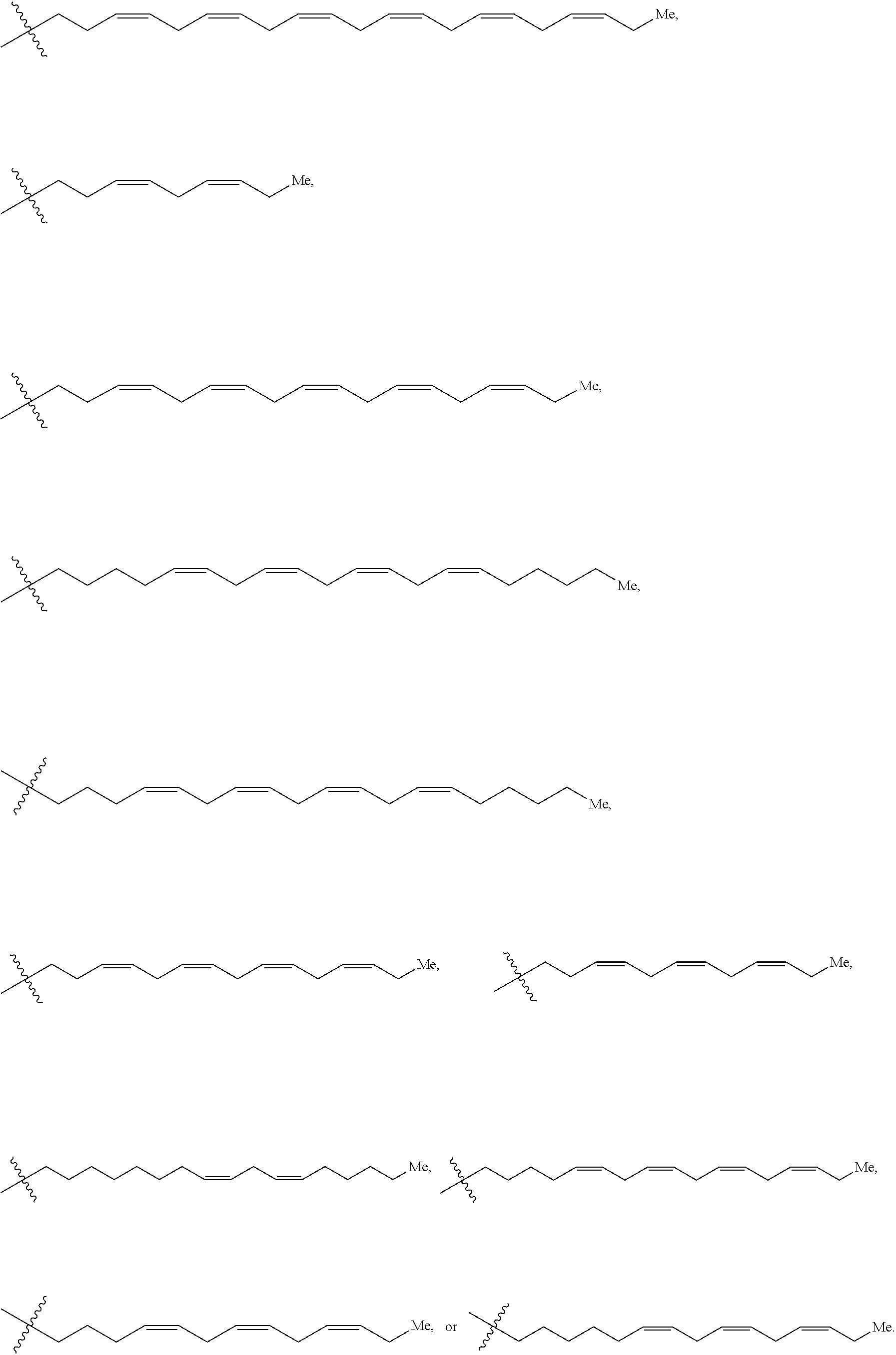

### PUFA Charged Group (“A”)

The PUFAs of the disclosure include a charged group (“A”). The charged group can be negatively charged or positively charged.

The negatively charged group can include a formula selected from D(CH₂)ₘNHC(O), or D(CH₂)ₘOC(O), or D(CH₂)ₘSC(O), wherein m is 1-6 (e.g., 1 or 2 or 3 or 4 or 5 or 6).

In some embodiments, A is D(CH₂)ₘNHC(O). In these embodiments, D can include COOH, SO₃H or OSO₃H. For example, m can be 1-3 and D can be COOH or SO₃H or OSO₃H.

In some embodiments, A is D(CHCH₃)ₘNHC(O). In these embodiments, D can include COOH, SO₃H or OSO₃H. For example, m can be 1-3 and D can be COOH or SO₃H or OSO₃H.

In some embodiments, A is D(CH₂)ₘOC(O). In these embodiments, D can include COOH, SO₃H or OSO₃H. For example, m can be 1-3 and D can be COOH or SO₃H or OSO₃H.

In some embodiments, A is D(CH₂)ₘSC(O). In these embodiments, D can include COOH, SO₃H or OSO₃H. For example, m can be 1-3 and D can be COOH or SO₃H or OSO₃H.

In cases wherein D is COOH, the PUFA can have a pKₐ in a lipid environment of less than about 7.4, or less than about 7.0, or less than about 6.5. Thus, PUFAs comprising COOH for D have a partial negative charge at physiological pH (i.e., pH 7.4). The PUFAs with a partial negative charge can shift the voltage dependence of the I_{Ks} channel up to about −5 mV, or up to about −10 mV, or up to about −15 mV, or up to about −20 mV, or up to about −25 mV, or up to about −30 mV, or up to about −35 mV, or up to about −40 mV, or up to about −45 mV, or up to about −50 mV at physiological pH. In some cases, the negatively charged PUFAs disclosed herein can shift the voltage dependence of the I_{Ks} channel in a range of about −5 mV to about −50 mV, or about −5 mV to about −30 mV, or about −10 mV to about −40 mV, or about −15 mV to about −30 mV, or about −20 mV to about −30 mV, or about −30 mV to about −50 mV. Thus, the partially negatively charged PUFAs can modulate the I_{Ks} channel, for example, at physiological pH or lower. For, example, the partially negatively charged PUFAs can activate the I_{Ks} channel, e.g., at physiological pH or lower.

In cases wherein D is SO₃H or OSO₃H, the PUFA can have a pKₐ in a lipid environment of less than about 6.5, or less than about 6.0, or less than about 5.5, or less than about 5.0. Thus, PUFAs comprising SO₃H or OSO₃H for D can have a permanent negative charge at physiological pH (i.e., pH 7.4). The PUFAs with a permanent negative charge can shift the voltage dependence of the I_{Ks} channel up to about −5 mV, or up to about −10 mV, or up to about −15 mV, or up to about −20 mV, or up to about −25 mV, or up to about −30 mV, or up to about −35 mV, or up to about −40 mV, or up to about −45 mV, or up to about −50 mV at physiological pH. In some cases, the negatively charged PUFAs disclosed herein can shift the voltage dependence of the I_{Ks} channel in a range of about −5 mV to about −50 mV, or about −5 mV to about −30 mV, or about −10 mV to about −40 mV, or about −15 mV to about −30 mV, or about −20 mV to about −30 mV, or about −30 mV to about −50 mV. Thus, the permanently negatively charged PUFAs can modulate the I_{Ks} channel, for example, at physiological pH or lower. For, example, the permanently negatively charged PUFAs can activate the I_{Ks} channel, e.g., at physiological pH or lower.

Examples of negatively charged PUFAs of the disclosure include:
N-arachidonoyl taurine (“N-AT”):

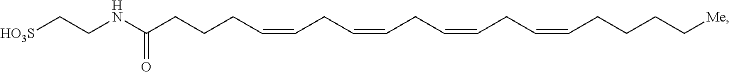 [SMILES: CCCCC/C=C\C/C=C\C/C=C\C/C=C\CCCC(=O)NCCS(=O)(=O)O]

docosahexaenoyl glycine(“DG” or “DHA-Gly”):

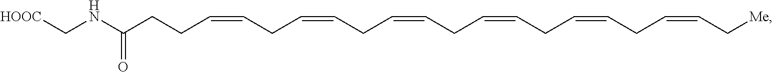 [SMILES: CC/C=C\C/C=C\C/C=C\C/C=C\C/C=C\C/C=C\CCC(=O)NCC(=O)O]

linoleoyl-glycine (“Lin-Gly”):

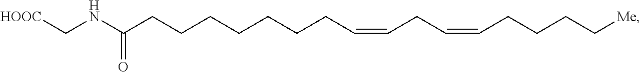 [SMILES: CCCCC/C=C\C/C=C\CCCCCCCC(=O)NCC(=O)O]

stearidonyl-glycine (“Ste-Gly”):

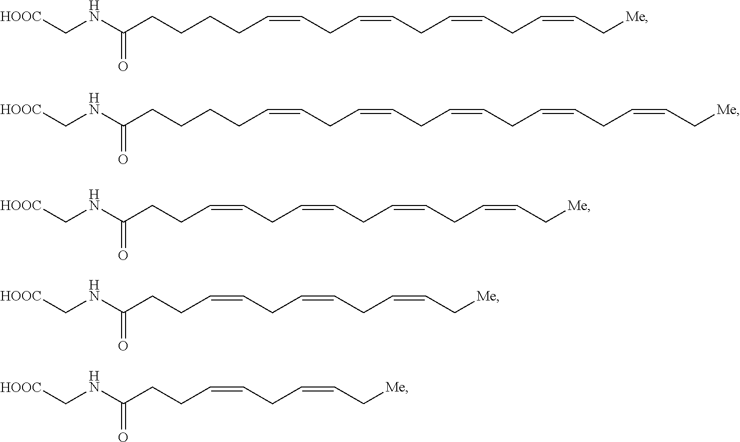 [SMILES: CC/C=C\C/C=C\C/C=C\C/C=C\CCCCC(=O)NCC(=O)O ; CC/C=C\C/C=C\C/C=C\C/C=C\C/C=C\CCCCC(=O)NCC(=O)O ; CC/C=C\C/C=C\C/C=C\C/C=C\CCC(=O)NCC(=O)O ; CC/C=C\C/C=C\C/C=C\CCC(=O)NCC(=O)O ; CC/C=C\C/C=C\CCC(=O)NCC(=O)O]

N-arachidonyl-alanine (“NAA”):

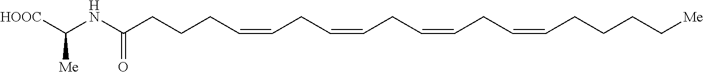 [SMILES: CCCCC/C=C\C/C=C\C/C=C\C/C=C\CCCC(=O)N[C@@H](C)C(=O)O]

5,8,11,14,17-all-cis-eicosapentaenoic acid (“EPA”)

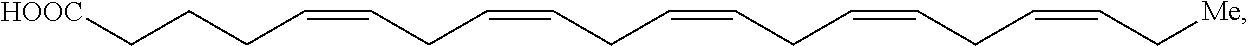 [SMILES: CC/C=C\C/C=C\C/C=C\C/C=C\C/C=C\CCCC(=O)O]

7,10,13-all-cis-hexadecatrienoic acid (“HTA”):

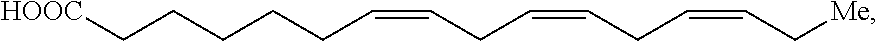 [SMILES: CC/C=C\C/C=C\C/C=C\CCCCCC(=O)O]

and
5,8,11-all-cis-tetradecatrienoic acid (“TTA”):

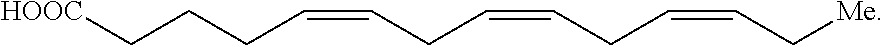 [SMILES: CC/C=C\C/C=C\C/C=C\CCCC(=O)O]

The positively charged group of the PUFA can include a formula selected from NH₂, NHR¹, N(R¹)₂, or N(R¹)₃⁺, wherein each R¹ independently is C₁₋₃ alkyl.

In some embodiments, A is NH₂.

In some embodiments, A is NHR¹. In these embodiments, A can include N(H)Me, N(H)Et, N(H)Pr, or N(H)iPr.

In some embodiments, A is N(R¹)₂. In these embodiments, A can include N(Me)₂, N(Me)Et, N(Me)Pr, N(Me)iPr, N(Et)₂, N(Et)Pr, N(Et)iPr, N(Pr)₂, N(Pr)iPr, or N(iPr)₂.

In some embodiments, A is N(R¹)₃⁺. In these embodiments, A can include N(Me)₃⁺, N(Me)₂(Et)⁺, N(Me)₂(Pr)⁺, N(Me)₂(iPr)⁺, N(Me)(Et)₂⁺, N(Me)(Pr)₂⁺, N(Me)(iPr)₂⁺, N(Et)₃⁺, N(Et)₂Pr⁺, N(Et)₂iPr⁺, N(Et)(Pr)₂⁺, N(Et)(iPr)₂⁺, N(Pr)₃⁺, N(Pr)₂(iPr)⁺, N(Pr)(iPr)₂⁺, or N(iPr)₃⁺. For example, A can be N(Me)₃⁺.

In cases wherein A is NH₂, NHR¹, or N(R¹)₂, the PUFA can have a pKₐ in a lipid environment of greater than about 8, or greater than about 8.5 or greater than about 9, or greater than about 9.5. Thus, PUFAs comprising NH₂, NHR¹, or N(R¹)₂ for A can have a partial positive charge at physiological pH (i.e., pH 7.4).

In cases wherein A is N(R¹)₃⁺, the PUFA can have a permanent positive charge.

PUFAs having a “A” group with a partial or permanent positive charge can shift the voltage dependence of the I_{Ks} channel up to about +5 mV, or up to about +10 mV, or up to about +15 mV, or up to about +20 mV, or up to about +25 mV, or up to about +30 mV, or up to about +35 mV, or up to about +40 mV, or up to about +45 mV, or up to about +50 mV at physiological pH. In some cases, the positively charged PUFAs disclosed herein can shift the voltage dependence of the I_{Ks} channel in a range of about +5 mV to about +50 mV, or about +5 mV to about +30 mV, or about +10 mV to about +40 mV, or about +15 mV to about +30 mV, or about +20 mV to about +30 mV, or about +30 mV to about +50 mV. Therefore, positively charged PUFAs can inhibit the I_{Ks} channel, e.g., at physiological pH.

For example, one contemplated positively charged PUFAs of the disclosure is arachidonyl amine (“AA⁺”):

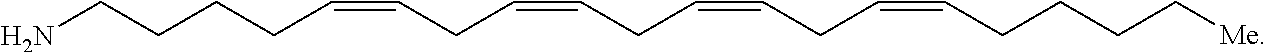 [SMILES: CCCCC/C=C\C/C=C\C/C=C\C/C=C\CCCCN]

### Synthesis of PUFAs

The PUFAs described herein are either commercially available, or can be synthesized by any method known to one skilled in the art. For example, 4,7,10,13,16,19-all-cis-docosahexaenoic acid (“DHA”), 4,7,10,13,16,19-all-cis-docosahexaenoic acid methyl ester (“DHA-Me”), 5,8,11,14,17-all-cis-eicosapentaenoic acid (“EPA”), and methyl 9-cis-octadecenoic acid (oleic acid) can be purchased from Sigma-Aldrich, and docosahexaenoyl glycine (“DG”) and N-arachidonoyl taurine (N-AT) can be purchased from Caymen Chemical.

Synthesis of arachidonyl amine was performed as described in S. I., Parkkari, T., Hammarstrom, S. & Elinder, F. Electrostatic Tuning of Cellular Excitability. Biophys J 98, 396-403 (2010) (see the Examples section). Synthesis of the acylated glycine compounds was performed by a two-step process described in Aneetha et al., Bioorganic & Medicinal Chemistry Letters, 19, 237-241 (2009). First, a fatty acid was conjugated with 1,2-dihydroxy-3-aminopropane using a combination of 1-ethyl-3-(3-dimethyllaminopropyl)carbodiimide hydrochloride (“EDC HCl”),N-hydroxybenzotriazole (“HOBT”), and triethylamine (“Et₃N”) in dichloromethane at room temperature. Second, the resulting diol was oxidized to an aldehyde using sodium periodate in a THF/H₂O mixture. The aldehyde was then oxidized to a carboxylic acid by a suitable means known in the art. Alternatively, the diol was oxidatively cleaved to directly form a carboxylic acid.

### Methods

As previously described, the disclosure provides PUFAs having a structure A-T, wherein A is a charged moiety and T is an alkenyl. The PUFAs described herein can shift the voltage dependence of the I_{Ks} channel towards more negative (if the PUFA is negatively charged) or more positive (if the PUFA is positively charged) membrane voltages, thereby activating or inhibiting the I_{Ks} channel, respectively.

Thus, one aspect of the disclosure relates to a method of modulating an I_{Ks} channel comprising contacting the I_{Ks} channel with a compound, A-T, in an amount effective to modulate the I_{Ks} channel, wherein: A is D(CH₂)ₘNHC(O), D(CHCH₃)ₘNHC(O), D(CH₂)ₘOC(O), D(CH₂)ₘSC(O), NH₂, NHR¹, N(R¹)₂, or N(R¹)₃⁺; D is CO₂H, SO₃H, or OSO₃H; T is C₅₋₂₉ alkenyl having at least two double bonds; each R¹ independently is C₁₋₃alkyl; and m is 1-6, as previously described above. The PUFAs disclosed herein are effective on Kv7.1/KCNE1 at physiological pH even in the presence of KCNE1.

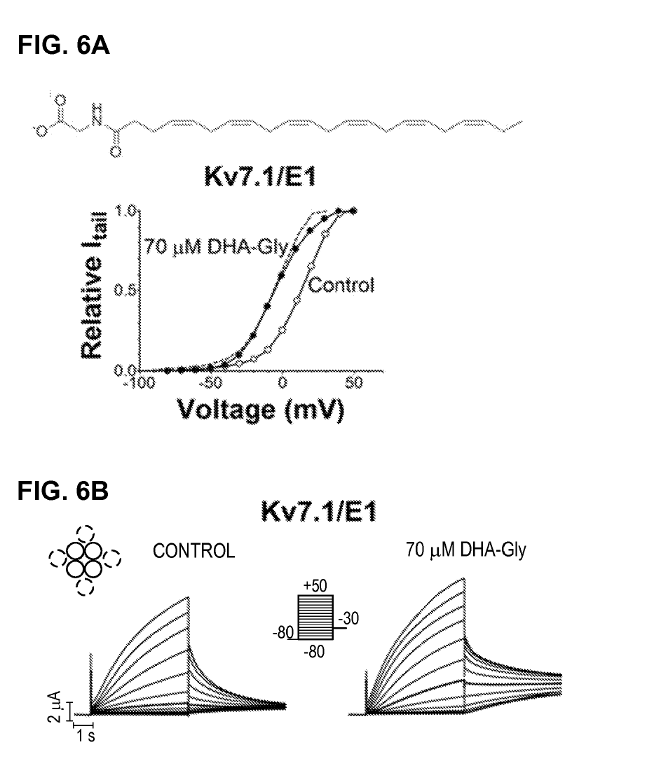

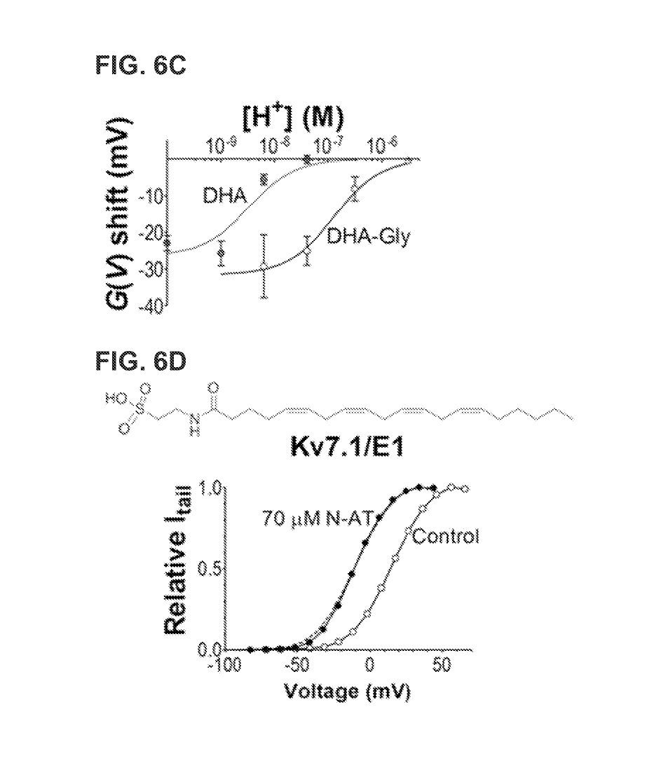

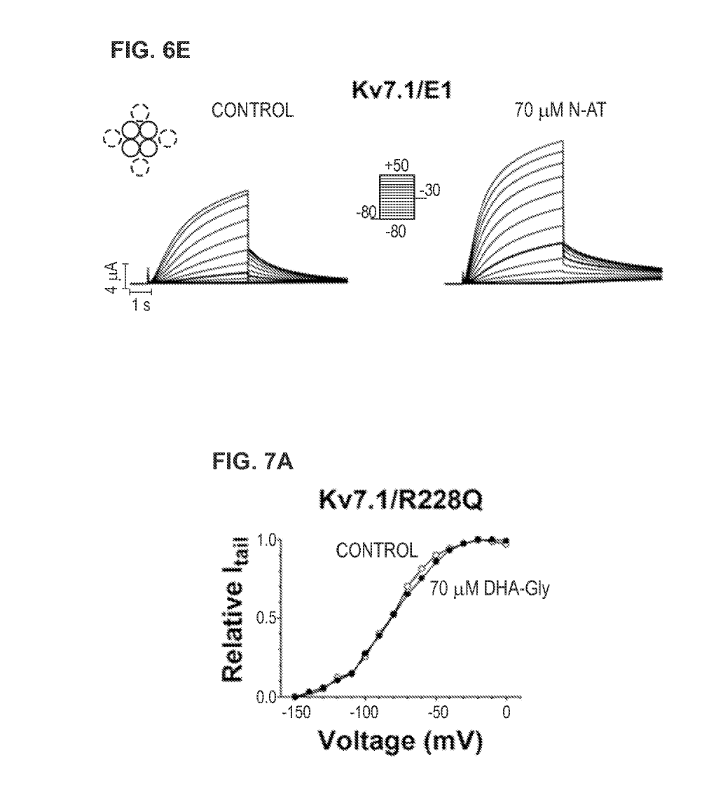

When Kv7.1/KCNE1 is contacted with a PUFA that has a partial negative charge (e.g., when D is COOH) or a permanent negative charge (e.g., when D is SO₃H or OSO₃H) at pH 7.4, the PUFA induces a negative shift in the G(V) curve (e.g., up to about −5 mV, or up to about −10 mV, or up to about −15 mV, or up to about −20 mV, or up to about −25 mV, or up to about −30 mV, or up to about −35 mV, or up to about −40 mV, or up to about −45 mV, or up to about −50 mV). For example, when Kv7.1/KCNE1 was contacted with docosahexaneoyl glycine (“DHA-Gly”) at pH 7.4, a negative G(V) shift of about −25 mV was induced (Example 3, FIG. 6A-C, Table 1). For example, when Kv7.1/KCNE1 was contacted with N-arachidonoyl taurine (N-AT) at pH 7.4, a negative G(V) shift of about −30 mV was induced (Example 3, FIG. 6D-E, Table 1).

When Kv7.1/KCNE1 is contacted with a PUFA that has a positive charge (e.g., when A is an amine) at pH 7.4, the PUFA induces a positive shift in the G(V) curve (e.g., up to about +5 mV, or up to about +10 mV, or up to about +15 mV, or up to about +20 mV, or up to about +25 mV, or up to about +30 mV, or up to about +35 mV, or up to about +40 mV, or up to about +45 mV, or up to about +50 mV). For example, when Kv7.1/KCNE1 was contacted with arachidonyl amine at pH 7, a positive G(V) shift of about +25 mV was induced (Example 3, FIG. 5).

The PUFAs disclosed herein can modulate an I_{Ks} channel by contacting the channel in vitro or in vivo. In some embodiments, the contacting occurs in vitro. In other embodiments, the contacting occurs in vivo. The PUFAs can contact an I_{Ks} channel in vivo by administering the PUFA to a subject in need of modulation of the I_{Ks} channel. Put another way, in various embodiments, the invention includes administering one or more PUFAs described herein to a subject, such as a human, in need thereof.

In some embodiments, the subject suffers from a condition resulting from an increase in the length of ventricular action potential, which corresponds to an increase of the QT interval of the ECG. Some of these conditions include, e.g., Long QT Syndrome, cardiac arrhythmia, atrial flutter, ventricular tachycardia, Romano-Ward syndrome, Jervell and Lange-Nielsen syndrome, or drug-induced Long QT syndrome. In these embodiments, the PUFA that is administered is negatively charged and activates the I_{Ks} channel. Thus, the negatively charged PUFA decreases the length of the ventricular action potential to reverse the effect of Long QT mutations.

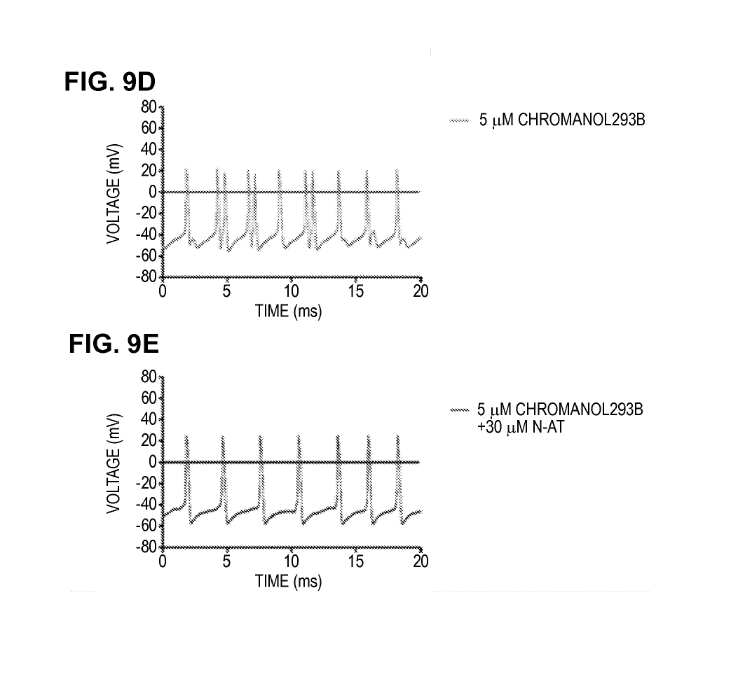

For example, the negatively charged PUFAs disclosed herein reduce the length of the ventricular action potential in cardiomyocytes. Rat cardiomyotes that were contacted with N-arachidonoyl taurine (“N-AT”) reduced the length of the ventricular action potential (see Example 6, FIG. 9A), and reversed the effect of Long QT mutations.

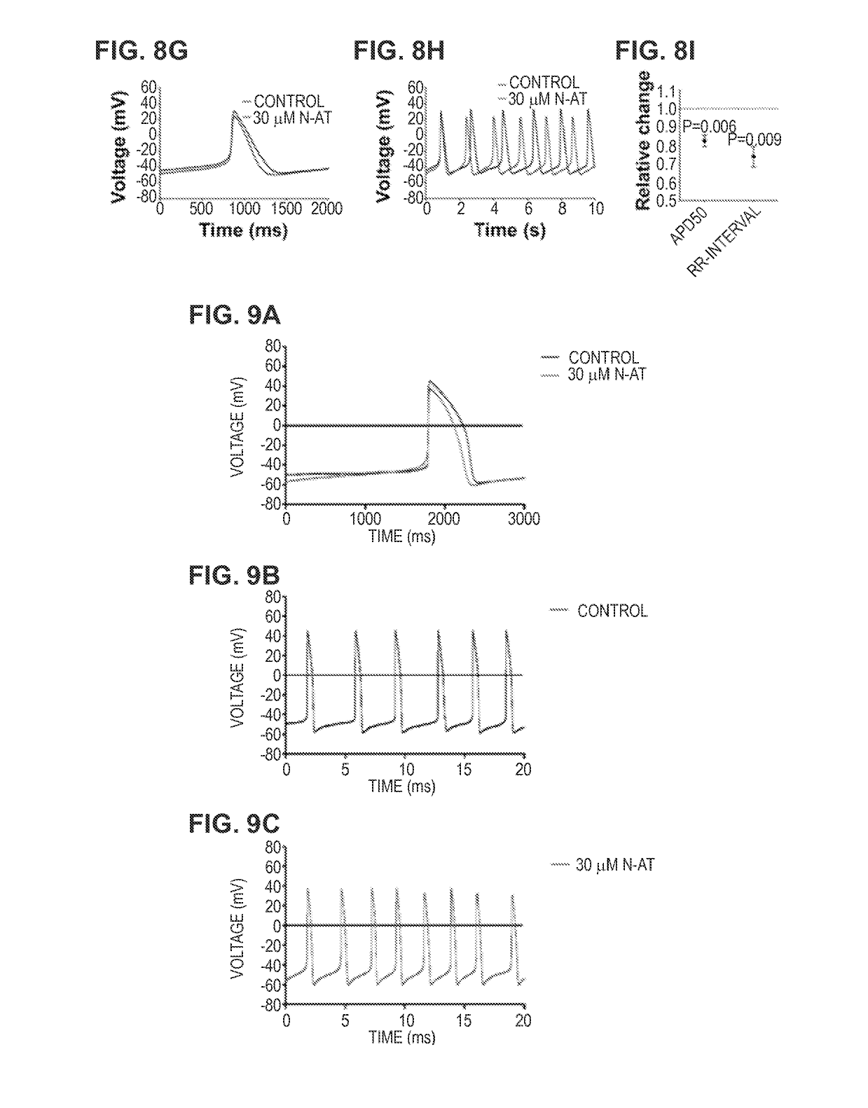

The negatively charged PUFAs also reverse the effects of arrhythmia in subjects. Rat cardiomyocytes in which arrhythmia had been induced were contacted with N-AT. The N-AT shortened the cardiomyocyte action potential duration, increased the frequency of action potential firing, and restored rhythmic firing (Examples 5-6, FIG. 8G-I, 9B-C, 9E, Table 2). N-AT had no effect on the resting potential, (Example 5, Table 2). Without being bound by any particular theory, the PUFAs disclosed herein activate more I_{Ks} channels that keep the cells hyperpolarized at the end of the action potential, preventing early after depolarizations (“EADs”) that are present in arrhythmias (see FIG. 9D-E).

N-AT and Chromanol are known to bind to different parts of the I_{Ks} channels (Chromanol is a pore blocker, whereas N-AT is binding to the voltage sensor). Without being bound by any particular theory, the effect of N-AT on cardiomyoctes is not just a competition with Chromanol to the same binding site. Rather, N-AT increases the currents by attracting the voltage sensor, whereas Chromanol is known to block the currents by binding to the pore. reversed the arrhythmia effect by shortening the cardiomyocyte action potential duration and increasing the frequency of action potential firing (Example 5, FIG. 8).

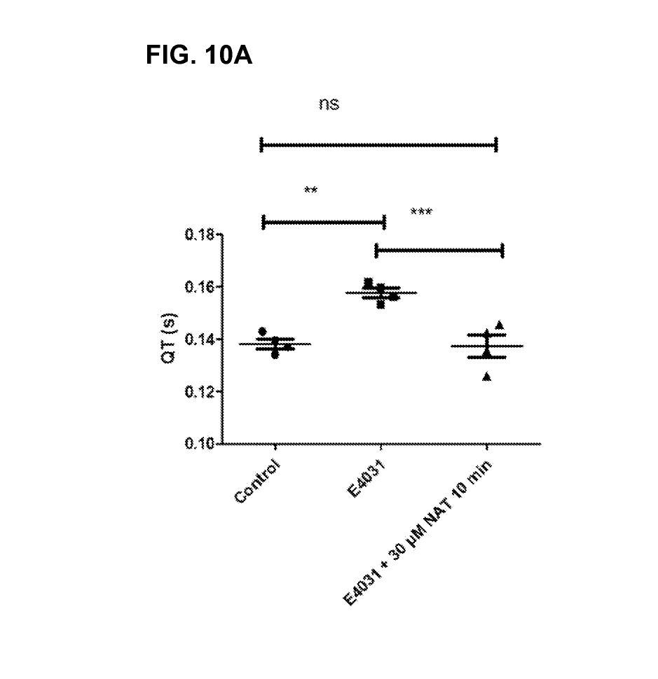

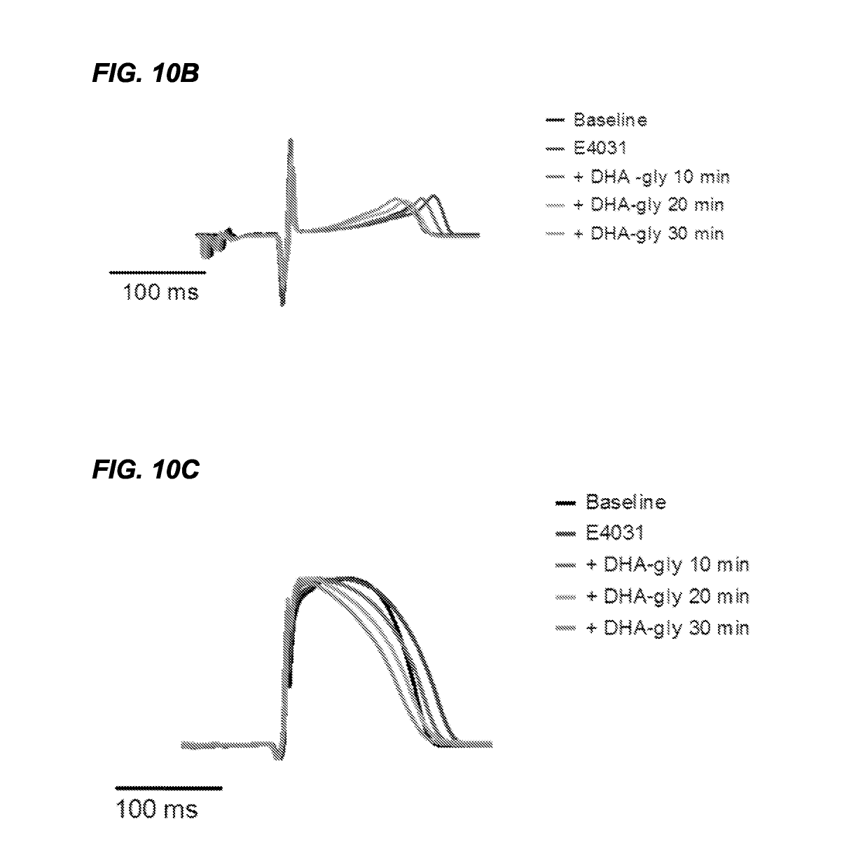

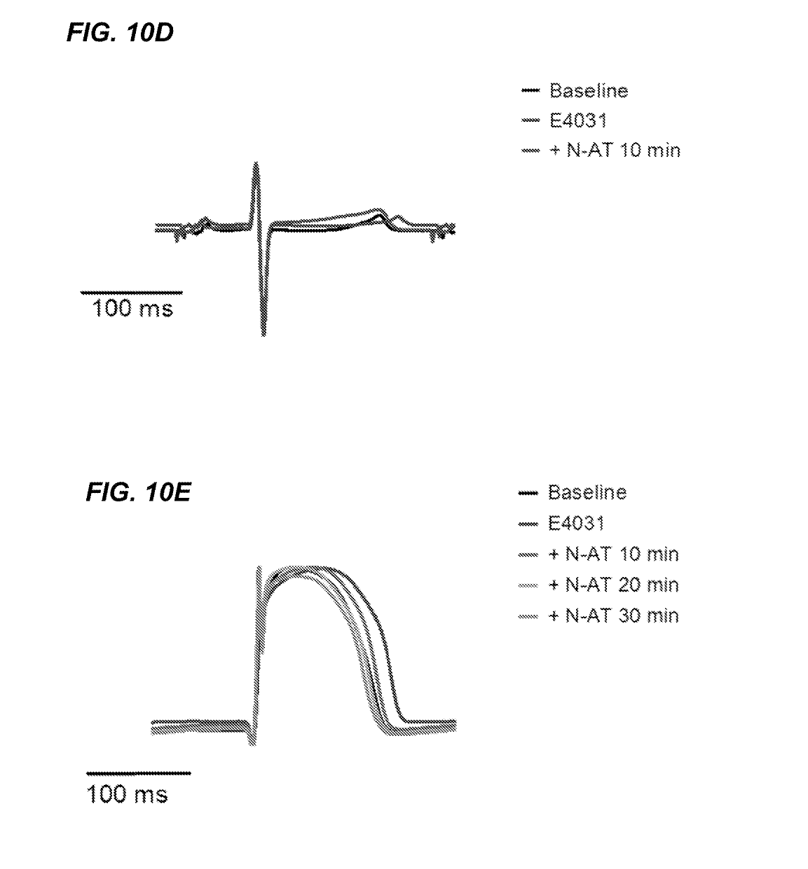

A number of blockbuster drugs have been taken off the market because of their tendency to induce Long QT syndrome in patients. The negatively charged PUFAs disclosed herein, however, have been found to prevent or treat drug-induced Long QT syndrome or Torsades de Pointes (TdP). For example, Long QT syndrome was induced in intact guinea pig hearts using the drug, E4031, a HERG channel blocker. N-AT and DHA-Gly were each able to restore the QT interval in the guinea pig hearts (Example 7, FIG. 10). Therefore, disclosed herein is a method for treating or preventing drug-induced Long QT syndrome through administration of a PUFA. Also disclosed herein, is a method for preventing drug-induced Long QT syndrome by co-administering a PUFA with a second therapeutic agent that has a tendency to induce Long QT syndrome. Examples of therapeutic agents capable of inducing QT prolongation and/or TdP include, but are not limited to, anti-infectives (e.g., clarithromycin, erythromycin, chloroquine, pentamidine, azithromycin, roxithromycin, telithromycin, moxifloxacin, and amantadine), anti-emetics (e.g., domperidone, cisapride, ondansetron, dolasetron, granisetron), anti-psychotics, (e.g., haloperidol, chlorpromazine, risperidone, quetiapine, sertindole, ziprasidone, lithium, clozapine), anti-depressants (e.g., escitalopram, venlafaxine), opioid analgesics (e.g., methadone), antihistamines (e.g., terfenadine), and anti-cancer drug (e.g., tamoxifen, nilotinib, lapatinib).

In view of the above, in various aspects, the invention includes a method of treating a condition resulting from an increase in the length of ventricular action potential in a subject. The method comprises administering to the subject a therapeutically effective amount of a PUFA described herein, such that the QT interval of the subject is decreased. Exemplary conditions resulting from an increase in the length of a ventricular action potential include, but are not limited to, Long QT Syndrome, cardiac arrhythmia, atrial flutter, ventricular tachycardia, Romano-Ward syndrome, Jervell and Lange-Nielsen syndrome, or drug-induced Long QT syndrome. Use of the PUFA described herein to treat a condition resulting from an increase in the length of ventricular action potential in a subject, as well as use of the PUFA described herein in the preparation of a medicament for treating the condition, are also contemplated.

In other embodiments, the subject suffers from a condition resulting from a decrease in the length of ventricular action potential, which corresponds to a decrease of the QT interval of the ECG. Some of these conditions include, e.g., Short QT Syndrome, blood clot formation, or atrial fibrillation. In these embodiments, the PUFA that is administered is positively charged and inhibits the I_{Ks} channel. Thus, the positively charged PUFAs disclosed herein can increase the length of the ventricular action potential to reverse the effect of Short QT mutations.

In view of the above, the invention further includes a method for treating a condition resulting from a decrease in the length of ventricular action potential in a subject. The method comprises administering to the subject a therapeutically effective amount of a PUFA described herein, such that the QT interval of the subject is increased. Exemplary conditions resulting from a decrease in the length of ventricular action potential include, e.g., Short QT Syndrome, blood clot formation, or atrial fibrillation. Use of the PUFA described herein to treat a condition resulting from a decrease in the length of ventricular action potential in a subject, as well as use of the PUFA described herein in the preparation of a medicament for treating the condition, are also contemplated.

Mechanistic effects of the PUFAs disclosed herein on the I_{Ks} channel are described in detail below.

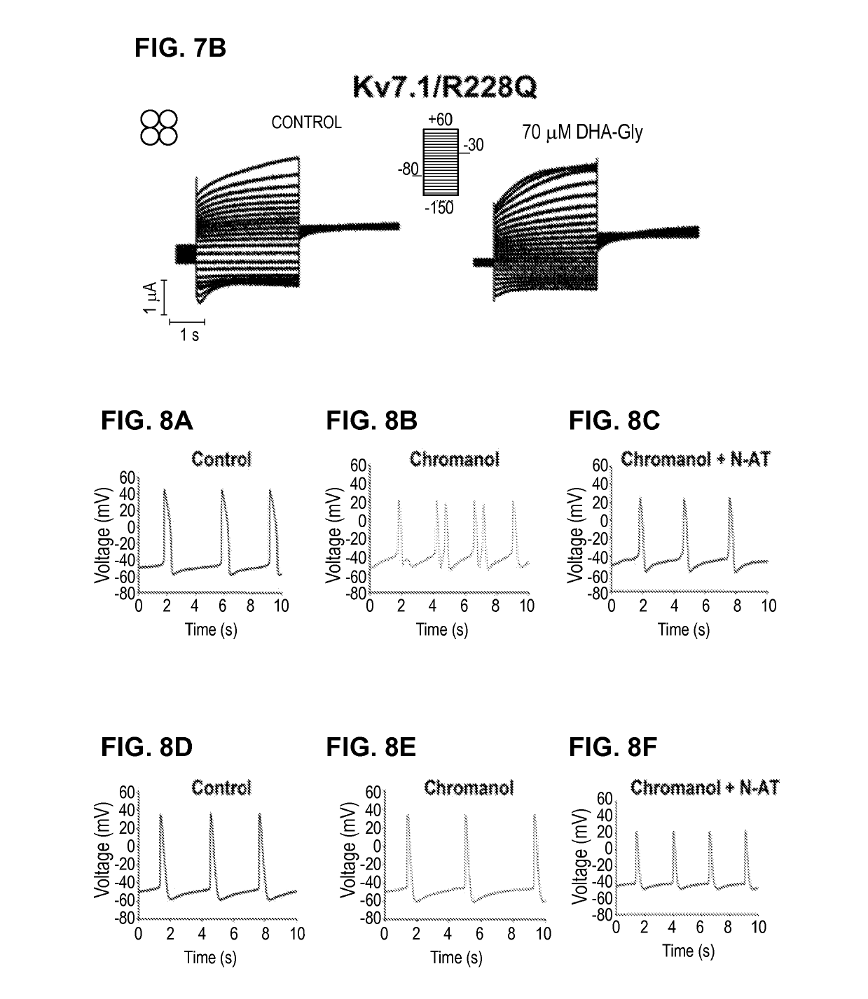

The PUFAs described herein can target the voltage sensing domain (“VSD”). For example, when the outermost positive gating charge R228 in S4 of Kv7.1 was neutralized, DHA-Gly did not shift the G(V) curve of R228Q (Example 4, FIG. 7A-B, Table 1). Therefore, PUFAs can electrostatically facilitate Kv7.1 channel opening via interaction with the extracellular-facing part of the positively charged voltage sensor S4. See also Börjesson et al., Biophys J 98, 396-403 (2010)).

The PUFAs described herein do not display gradual potency depending on the number of KCNE1 subunits in the channel complex (e Example 2, FIG. 4G-J, Table 1), in contrast to the previously reported Kv7.1 channel modulators R-L3 and ML277 (Salata, J. J. et al. Mol Pharmacol 54, 220-230 (1998); Yu, H. et al. Proc Natl Acad Sci USA 110, 8732-8737 (2013); Gao et al., J Biol Chem 283, 22649-22658 (2008)). Without being bound by any particular theory, PUFAs can target the channel's VSD, whereas ML277 and R-L3 have been suggested to affect the pore domain of Kv7.1.

The effect of PUFAs on Kv7.1/KCNE1 depends on the external pH of the channel. For example, at pH 9, DHA was able to shift the G(V) curve of Kv7.1/KCNE1 in the negative direction, similar to the effects of DHA on Kv7.1 alone (Example 2, FIG. 4C-D, K).

Further guidance for using the compound A-T to contact a I_{Ks} channel can be found in the Examples section, below.

### Pharmaceutical Formulations

Also provided herein are pharmaceutical formulations that include the compound A-T, as previously described herein, and one or more pharmaceutically acceptable excipients.

The PUFA described herein can be administered to a patient in a therapeutically effective amount. The PUFA can be administered alone or as part of a pharmaceutically acceptable composition or formulation. In addition, the PUFA can be administered all at once, as for example, by a bolus injection, multiple times, e.g. by a series of tablets, or delivered substantially uniformly over a period of time, as for example, using transdermal delivery. It is also noted that the dose of the compound can be varied over time.

The PUFA disclosed herein can be administered in combination with one or more additional pharmaceutically active compounds/agents. The additional pharmaceutically active compounds/agents may be traditional small organic chemical molecules or can be macromolecules such as a proteins, antibodies, peptibodies, DNA, RNA or fragments of such macromolecules.

The PUFA disclosed herein and other pharmaceutically active compounds, if desired, can be administered to a patient by any suitable route, e.g. orally, rectally, parenterally, (for example, intravenously, intramuscularly, or subcutaneously) intracisternally, intravaginally, intraperitoneally, intravesically, or as a buccal, inhalation, or nasal spray. The administration can be to provide a systemic effect (e.g. eneteral or parenteral). All methods that can be used by those skilled in the art to administer a pharmaceutically active agent are contemplated.

The PUFAs described herein exhibit good permeability in differentiated Caco-2 cells in vitro (see Example 9), thus indicating a likelihood to exhibit good in vivo absorption across the gut wall.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The PUFA described herein can be administered to a patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the patient, the severity of the condition or disease being treated, and the pharmacological activity of the compound being administered. The determination of dosage ranges and optimal dosages for a particular patient is within the ordinary skill in the art.

When a patient is to receive or is receiving multiple pharmaceutically active compounds, the compounds can be administered simultaneously, or sequentially. For example, in the case of tablets, the active compounds may be found in one tablet or in separate tablets, which can be administered at once or sequentially in any order. In addition, it should be recognized that the compositions may be different forms. For example, one or more compound may be delivered via a tablet, while another is administered via injection or orally as a syrup. All combinations, delivery methods and administration sequences are contemplated.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Examples

The following examples are provided for illustration and are not intended to limit the scope of the invention

### Molecular Biology

cDNAs encoding human Kv7.1 (GEnBAnk Acc. No. NM_000218) and KCNE1 (NM_000219) were subcloned into the expression vectors pGEM or pXOOM, containing the 5′- and 3′-untranslated regions for Xenopus laevis β-globin as well as a poly-A segment (Jespersen et al., Biotechniques 32, 536-538, 540 (2002)). The truncated KCNE1 construct KCNE1ΔC was constructed by introducing a Stop codon at position 67 using Quikchange site-directed mutagenesis kit (Qiagen). The construct was fully sequenced to ensure proper mutation and absence of unwanted mutations (sequencing by Genewiz). The concatemers E1-Kv7.1-Kv7.1 and E1-Kv7.1 were provided by Dr. R. S. Kass at Columbia University, and can be prepared as described in Chan et al. Journal of General Physiology 132(2), 135-144, (2012)). The E1-Kv7.1 construct was made by linking the C-terminus of KCNE1 to the N-terminus of KCNQ1. The E1-Kv7.1-Kv7.1 construct was made by digesting the E1-Kv7.1 and a Kv7.1-Kv7.1 dimer with Xho1 and inserting the 2-kb fragment from the Kv7.1-Kv7.1 dimer digest into the cut KCNE1-Kv7.1 construct. The DNA encoding E1-Kv7.1-Kv7.1 and E1-Kv7.1 were in the pGEM vector and contained mutations to make the constructs suitable for voltage-clamp fluorometry recordings (Wang et al., J Biol Chem 273, 34069-34074 (1998)). The original KCNE1 and Kv7.1 sequences were therefore removed, one at a time, from the pGEM vector using different restriction enzymes (New England Biolabs). Sequences were separated using gel electrophoresis and harvested using Qiagen gel extraction kit. Corresponding wild type sequences were ligated into the construct using T4 Ligase (New England Biolabs). To ensure correct sequence the constructs were sequenced (Genewiz) and tested with different restriction enzymes in combination with gel electrophoresis to measure length and conformation of the gene. cRNA for injection of all constructs was prepared from linearized DNA using the T7 mMessage mMachine transcription kit (Ambion) according to the manufactures instructions. RNA quality was checked by gel electrophoresis, and RNA concentrations were quantified by UV spectroscopy.

Xenopus laevis Oocytes

Xenopus laevis oocytes were isolated and maintained (Bentzen, B. H. et al. Neuropharmacology 51, 1068-1077 (2006)). Approximately 50 nl cRNA (50 ng Kv7.1, KCNE1-Kv7.1-Kv7.1 or KCNE1-Kv7.1 alternatively 25 ng Kv7.1 together with 8 ng KCNE1 or KCNE1ΔC) was injected into each oocyte using a Nanojet microinjector (Drummond). Electrophysiological experiments were made 2-5 days after injection. Currents were measured at room temperature with the two-electrode voltage-clamp technique (CA-1B Dagan amplifier). Currents were sampled at 1-3.3 kHz, filtered at 500 Hz, and not leakage corrected. Recording electrodes were pulled from borosilicate glass, filled with 3 M KCl, and had a resistance of 0.3-2.0 MΩ. A control solution containing (in mM): 88 NaCl, 1 KCl, 15 HEPES, 0.4 CaCl₂, and 0.8 MgCl₂ was used. NaOH was added to adjust pH to 7.4, yielding a final sodium concentration of about 100 mM. The holding voltage was generally set to −80 mV. Activation curves were generally elicited by stepping to test voltages between −110 and +60 mV (2-4 s durations and 10 mV increments) followed by a tail voltage of −30 mV. For recordings of Kv7.1 at pH 9 and pH 10, the holding voltage was set to more negative voltages because the largest DHA-induced shifts induced channel opening already at −80 mV. In addition to effects on channel voltage dependence, arachidonyl amine induced Kv7.1 channel inactivation. Therefore, in arachidonyl amine experiments a brief hyperpolarizing pulse of 50 ms to −120 mV was introduced between the test voltage and the tail voltage to relief the channels from inactivation.

### Rat Cardiomyocytes

On gestation day (GD) 16 of pregnancy time-mated Sprague Dawley rats (Taconic, Denmark) were sacrificed in a CO₂ chamber followed by cervical dislocation. Cell preparation of cardiomyocytes and culturing on glass coverslips were made (Danielsson, C. et al. Cardiovasc Res 97, 23-32 (2013)). Spontaneous action potentials were measured using the patch-clamp technique, whole cell configuration, 1-3 days after culturing. An Axopatch 200B patch-clamp amplifier, Digidata 1140A converter (Molecular Devices) and pClamp software (Molecular Devices) were used to acquire data. The extracellular solution contained (in mM): 140 NaCl, 5.4 KCl, 1 MgCl₂, 1.8 CaCl₂, 10 Glucose and 10 HEPES (pH 7.4, adjusted with NaOH). The intracellular solution contained (in mM): 130 K-gluconate, 9 KCl, 8 NaCl, 10 EGTA, 1 MgCl₂, 10 HEPES and 3 MgATP (pH 7.3, adjusted with KOH). The solutions were perfused through the recording chamber (flow: 0.5 mL/min). All chemicals were purchased from Sigma-Aldrich. The patch pipettes, made of borosilicate glass, had a resistance between 4-6 MΩ, once filled with intracellular solution. All experiments were carried out at 35° C. To analyze action potential characteristics a stable 60-seconds recording was selected before and after application of the test solution. The average data were then calculated for the complete trace in Clampfit 10 (Molecular Devices). Liquid junction potential was not adjusted for.

### Test Compounds

4,7,10,13,16,19-all-cis-docosahexaenoic acid (DHA), 4,7,10,13,16,19-all-cis-docosahexaenoic acid methyl ester (DHA-me), 5,8,11,14,17-all-cis-eicosapentaenoic acid (EPA), and methyl 9-cis-octadecenoic acid (oleic acid) were purchased from Sigma-Aldrich. Docosahexaenoyl glycine and N-arachidonoyl taurine were purchased from Cayman Chemical. Synthesis of arachidonyl amine (Scheme 1) was performed as described Börjesson et al. Cell Biochem Biophys 52, 149-174 (2008), S. I., Parkkari, T., Hammarström, S. & Elinder, F. Electrostatic Tuning of Cellular Excitability. Biophys J 98, 396-403 (2010). Arachidonic acid (Nu Chek prep, Inc) was used as a starting material to produce arachidonyl alcohol. For synthesis of arachidonyl alcohol, LiAlH₄ (0.5 g, 13 mmol) was suspended into dry tetrahydrofuran (THF, 50 mL) and the mixture was cooled on an ice bath. Arachidonic acid (1.1 g, 3.5 mmol) in dry THF (40 mL) was added slowly. After the addition, stirring was continued for 1 hour on the ice bath and an additional hour at room temperature. Ice cold water (1 mL) following 10% NaOH (0.2 mL) was added slowly and stirring was continued for 1 hour. The mixture was filtered through Celite and the filtrate was dried over Na₂SO₄. Evaporation of solvents gave 800 mg (80%) of a colorless, oily product. ¹HNMR (CDCl₃): δ 0.89 (t, ³J=3.6 Hz, 3H), 1.19 (t, ³J=2.5 Hz, 1H) 1.25-1.39 (m, 6H), 1.42-1.48 (m, 2H), 1.57-1.62 (m, 2H), 2.04-2.13 (m, 4H), 2.80-2.85 (m, 6H), 3.66 (q, ³J=6.0 Hz, 2H), 5.31-5.43 (m, 8H).

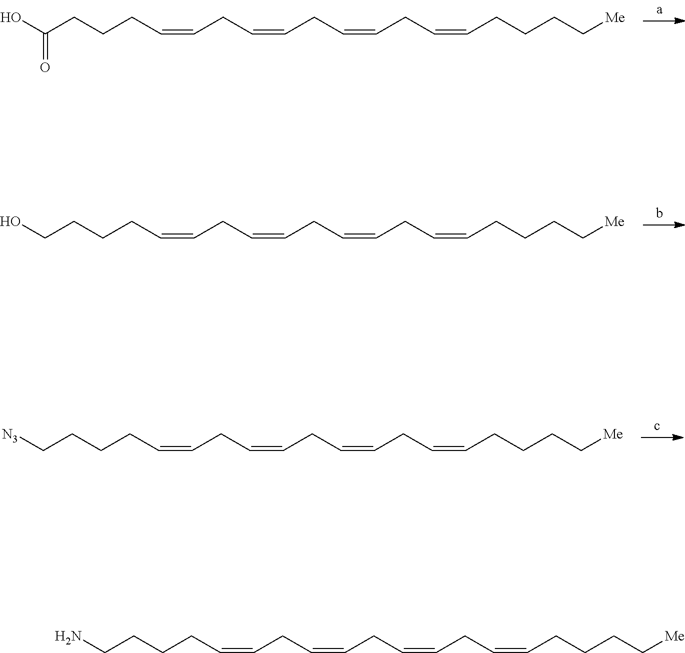 [SMILES: CCCCC/C=C\C/C=C\C/C=C\C/C=C\CCCC(=O)O ; CCCCC/C=C\C/C=C\C/C=C\C/C=C\CCCCO ; CCCCC/C=C\C/C=C\C/C=C\C/C=C\CCCCN=[N+]=[N-] ; CCCCC/C=C\C/C=C\C/C=C\C/C=C\CCCCN]

Scheme 1. (a) 1. LiAlH₄, THF, 2. H₂O, 10% NaOH; (b) pyridine, methanesulphonyl chloride, 2. DMF, NaN₃; (c) LiAlH₄, THF, diethyl ether.

Test compounds were dissolved in 99.5% ethanol to a concentration of 100 mM and stored at −20° C. (Börjesson et al., Biophys J 95, 2242-2253 (2008); Börjesson et al., Biophys J 98, 396-403 (2010)). Shortly before experiments, test compounds were diluted in control solution to the desired test concentration. Pure control solution was added to the bath using a gravity-driven perfusion system. To avoid binding of test compounds to the perfusion system, test solutions were added manually with a glass Pasteur pipette. The added volume was enough to replace the bath solution manifold. Previously, the effective concentration of fatty acids was determined to be 70% of the nominal concentration due to binding to the Perspex chamber (Börjesson 2010). As the present chamber has a similar design and is in the same material, the binding was assumed to be similar. All fatty acid and fatty acid analogue concentrations mentioned in this work are the estimated effective concentrations, i.e., 70% of the nominal concentration. Generally, the test compound effect was poorly reversible. The PUFA effect could not be washed away with pure control solution. When adding 100 mg/L albumin during washing the recovery was much improved however not complete.

To avoid possible cyclooxygenase enzyme metabolization of arachidonyl amine, oocytes were pretreated with 1 μM indomethacin and 1 μM indomethacin was supplemented to the recording solutions as described in (Börjesson 2010).

### Electrophysiological Analysis

Xenopus laevis oocyte data were acquired using either Pulse software (HEKA) or Clampex 10.2 (Axon Instruments), and analyzed with Clampfit 10 (Axon Instruments) and GraphPad Prism 4 (GraphPad Software). To quantify effects on the voltage dependence, tail currents (measured shortly after initiation of repolarization) were plotted against the prepulse (test) voltage. The following Boltzmann̂4 was fitted to the control curve and the given slope factor was noted

G(V)=A/(1+exp((V_{1/2}−V)/s))⁴, (1)

where V_{1/2} is the midpoint and s the slope factor. The same Boltzmann̂4 was fitted to the test substance Iₜₐᵢₗ vs voltage curve with the slope factor constrained to the value given for the control curve. The V_{1/2} values were compared to quantify the shift in voltage dependence. For illustrative clarity in figures showing Iₜₐᵢₗ vs voltage, the curves were instead normalized with the smallest value set to 0 and the largest value set to 1. The curves shown in the figures are not Boltzmann fits but simply lines connecting the data points. For Kv7.1 recordings in pH 9 and 10 with DHA concentrations ≧21 μM the conductance was instead calculated from the steady-state current (Iₛₛ) at the end of the test pulse as:

G(V)=Iₛₛ/(V−V_{K}) (2)

where V is the absolute membrane voltage and V_{K} the reversal potential for the K ion (−100 mV). This was because the pH-induced shift in combination with the large DHA-induced shift of voltage dependence compromised the tail currents.

To quantify the concentration dependence and pH dependence of the DHA-induced shift of voltage dependence, ΔV, the following equation was used:

ΔV=ΔVₘₐₓ/(1+(c_{0.5}/c)), (3)

where ΔVₘₐₓ is the maximal shift, c_{0.5} the concentration causing 50% of the maximal shift, and c the concentration of DHA (or

### Statistical Analysis

Average values are expressed as mean±SEM. Mean values for shifts in voltage dependence were analyzed using a two-tailed one samplet-test where mean values were compared with a hypothetical value of 0 or where mean values were compared to each other. Effects of N-AT on action potential peak amplitude and RMP in cardiomyocytes were analyzed using paired two-tailed t-test. Effects of N-AT on the relative change of APD and RR-interval were analyzed using two-tailed one samplet-test where mean values were compared with a hypothetical value of 1. P<0.05 is considered as statistically significant.

### Example 1: PUFAs Open Kv7.1 Channels in the Absence of KCNE1

Extracellular application of the docosahexaenoic acid (DHA) in the concentration range of circulating plasma PUFAs (Fraser, D. D. et al. Neurology 60, 1026-1029 (2003)) shifts the conductance versus voltage (G(V)) curve of human Kv7.1 in the negative direction along the voltage axis (FIG. 2A-B) without affecting maximal conductance (FIG. 2D). 70 μM DHA shifts the G(V) by −9.3±0.9 mV (n=3), and 7 μM DHA induces significant G(V) shifts (−3.0±0.5 mV, n=3, P=0.04). The estimated maximum shift is −15.8±2.3 mV and 50% of the maximum shift is caused by 50±20 μM DHA (FIG. 2C). See also Table 1.

The effect of PUFAs having slightly different structures on Kv7.1 also was tested. Eicosapentaenoic acid (“EPA”), which has a shorter tail than DHA, had an effect similar to DHA on Kv7.1. Oleic acid, which is monounsaturated, had no effect on Kv7.1. DHA methyl ester (70 μM), which has no charged group, had no effect on the voltage dependence of Kv7.1. Arachidonyl amine (70 μM), which has a positively charged group, shifted the G(V) curve by +9.7±2.1 mV (n=8) (FIG. 3A, Table 1).

The effect of pH on PUFAs also was tested. Unsaturated fatty acids in a lipid environment are protonated/deprotonated with an apparent pKₐ of about 7 (Börjesson et al., Biophys J 95, 2242-2253 (2008); Hamilton, J. A. J Lipid Res 39, 467-481 (1998)). See FIG. 3B. The apparent pKₐ value for DHA on Kv7.1 is about 7.7 as reported by the shift of the G(V) relation (FIG. 3C). Increasing external pH from 7.4 to 9 or 10 increases the shift in negative direction of the G(V) curve protocol by DHA, whereas reducing external pH to 6.5 abolishes the DHA effect (FIG. 3D).

The effect of co-application of DHA and DHA methyl ester also was tested. Co-application of 70 μM DHA and 70 μM DHA methyl ester reduces the shift of the G(V) relation (−5.5±0.6 mV, n=4) compared to application of 70 μM DHA alone (−9.3±0.9 mV) (FIG. 3E-F), indicating competition of these compounds for the site of DHA action. The induced G(V) shift from co-application of 70 μM DHA and 70 μM DHA methyl ester (−5.5 mV) is close to that produced by 35 μM DHA alone (−6.5 mV) (FIG. 3G). Thus, while the uncharged DHA methyl ester competes with DHA for binding, it has by itself no effect on Kv7.1 channel activation.

### Example 2: KCNE1 Reduces PUFA Effect on Kv7.1 Channels

Kv7.1 was co-expressed with high concentrations of KCNE1, and the DHA sensitivity of the heteromeric Kv7.1/KCNE1 channel was tested. The GO shift of Kv7.1 induced by 70 μM DHA is not observed when Kv7.1 is co-expressed with KCNE1 (FIG. 4A-B, Table 1).

Kv7.1 was co-expressed with a KCNE1 construct in which the C-terminus was truncated (KCNE1ΔC). This construct was previously shown to associate with Kv7.1, but to alter channel kinetics and voltage dependence only to a small degree (Tapper et al., J Gen Physiol 116, 379-390 (2000)). Kv7.1/KCNE1ΔC displays kinetics and activation voltage dependence similar to Kv7.1 alone (FIG. 4E-F). The Kv7.1/KCNE1ΔC channel is however insensitive to 70 μM DHA (FIG. 4E-F, Table 1). A strong alteration of activation gating by KCNE1 is, thus, not required for suppression of DHA action.

KCNE1 has been shown to eliminate the effect of Kv7.1 activators, such as R-L3 and ML277. These compounds potentiate Kv7.1 homotetrameric channels expressed in Xenopus oocytes, but not Kv7.1 co-expressed with high levels of KCNE1 (Salata, J. J. et al. Mol Pharmacol 154, 220-230 (1998); Yu, H. et al. Proc Natl Acad Sci USA 110, 8732-8737 (2013)). A gradual decrease in the potency of these compounds is observed depending on the number of KCNE1 subunits in the Kv7.1/KCNE1 channel complex. Therefore, tests were performed to determine whether the potency of DHA on Kv7.1/KCNE1 channels increases if the number of KCNE1 subunits in the Kv7.1/KCNE1 complex is reduced.

Two concatemers were constructed, KCNE1-Kv7.1-Kv7.1 and KCNE1-Kv7.1, forcing either a 4:2 or a 4:4 stoichiometry of Kv7.1 and KCNE1 subunits in assembled channels. 70 μM DHA applied to either one of the concatemer channels only shifted the G(V) relation by a small positive voltage (FIG. 4G-J). This positive shift (observed at external pH 7.4) was unique for the concatemer channels, because it is not seen in wild type Kv7.1/KCNE1 channels (Table 1). This positive shift did not require the negative charge of the DHA because the uncharged DHA methyl ester induces a similar positive shift in both concatemer channels (Table 1). Experiments on these concatemer channels show that a Kv7.1 channel with only two KCNE1 subunits is not more sensitive to DHA than a channel with four KCNE1 subunits.

The effect of local proton concentration in the DHA binding site on the potency of DHA on Kv7.1/KCNE1 at pH 9 was tested. In contrast to the experiments above pH 7.4, at pH 9 the G(V) curves of WT Kv7.1 co-expressed with KCNE1 (FIG. 4C-D, Table 1) and of both concatemer channels (Table 1) were shifted by about −30 mV when 70 μM DHA was applied. These shifts are comparable to the DHA-induced G(V) shift of Kv7.1 alone at pH 9 (Table 1). Consistent with this view, the DHA effect on channel activation appears at higher pH in channels formed by Kv7.1/KCNE1 than in channels formed by Kv7.1 alone (FIG. 4K).

### Example 3: Analogs of PUFAs are Effective on Kv7.1/KCNE1 Channels

The effect of KCNE1-induced protonation on amino-analogs of PUFAs was tested. 70 μM arachidonyl amine induced a large shift in the G(V) curve of the Kv7.1/KCNE1 channel towards positive voltages (FIG. 5A-B). The average shift was +22.6±1.9 mV (n=10). The G(V) shift induced by 70 μM arachidonyl amine at pH 7.4 was about twice that seen in channels formed by Kv7.1 alone (+22.6±1.9 compared with +9.7±2.1 mV, FIG. 5C, Table 1). The opposite effects of KCNE1 co-expression on DHA and arachidonyl amine sensitivities (FIG. 5C) suggests that KCNE1 modulates the PUFA site of the Kv7.1 channel by lowering the local pH.

The effect of PUFA having a lowered pKₐ, docosahexaenoyl glycine (“DHA-Gly”), on the Kv7.1 channel was tested. 70 μM DHA-Gly shifts the G(V) curves of both channels formed by Kv7.1 alone (Table 1) and formed from Kv7.1/KCNE1 (FIG. 6A-B) by similar amounts (Table 1). DHA-Gly has an apparent pKₐ of 6.9 for Kv7.1/KCNE1, about 1.5 pH unit lower than that of DHA (FIG. 6C).

The effect of a permanently charged PUFA on the Kv7.1 channel was tested. 70 μM of the permanently charged N-arachidonoyl taurine shifted the G(V) curves of both channels formed by Kv7.1 alone and formed from Kv7.1/KCNE1 by similar amounts (Table 1, FIG. 6D-E). Thus, even in the presence of KCNE1, Kv7.1 is activated by a PUFA analogue with a lower pKₐ value and a PUFA analogue with a permanent charge.

The G(V) shifts of (i) Kv7.1 induced by 70 μM DHA at pH 9 and 10 (almost fully deprotonated), of (ii) Kv7.1/KCNE1 by 70 μM DHA at pH 9 (almost fully deprotonated), of (iii) Kv7.1 and Kv7.1/KCNE1 by 70 μM DHA-Gly at pH 7.4 (almost fully deprotonated), and of (iv) Kv7.1 and Kv7.1/KCNE1 by 70 μM N-arachidonoyl taurine at pH 7.4 (negatively charged) are all about −30 mV, suggesting that, as long as the PUFA is charged, the effect of the PUFA is independent of KCNE1.

### Example 4: PUFAs Target the Voltage Sensor Domain (“VSD”)

The mechanism by which PUFAs electrostatically facilitate Kv7.1 channel opening was tested. The outermost positive gating charge R228 in S4 of Kv7.1 was neutralized by making the R228Q mutation. 70 μM DHA-Gly did not shift the G(V) curve of R228Q (FIG. 7A-B, Table 1). Neutralizing the outermost positive gating charge in S4 abolished the effect of DHA-Gly on Kv7.1. Therefore, PUFAs electrostatically facilitate Kv7.1 channel opening via interaction with the extracellular-facing part of the positively charged voltage sensor S4.

### Example 5: Effect of N-Arachidonoyl Taurine on Cardiomyocytes

The effect of N-arachidonoyl taurine on isolated embryonic rat cardiomyocytes was tested. A sub-saturating concentration of the I_{Ks} channel blocker Chromanol 293B, which is known to induce arrhythmia (Aiba, T. et al. J Am Coll Cardiol 45, 300-307 (2005)), was applied to the cardiomyocytes. In general, two types of effects are seen upon Chromanol 293B application: (1) arrhythmic firing (FIG. 8A-B) or (2) a slowing of the frequency of spontaneous action potential firing (FIG. 8D-E). The different effects are most likely due to different subtypes of cardiomyocytes. Independent of the type of effect induced by Chromanol 293B application, 30 μM N-arachidonoyl taurine reversed the effect, abolishing the arrhythmic firing (FIG. 8C) or increasing the frequency of spontaneous action potential firing (FIG. 8F). The anti-arrhythmic effect of N-arachidonoyl taurine was associated with a shortening of the cardiomyocyte action potential duration and an increase in the frequency of action potential firing (FIG. 8G-I, Table 2). 30 μM N-arachidonoyl taurine had no effect on the resting membrane potential (Table 2) but decreased the action potential peak amplitude by −9±1 mV (Table 2).

### Example 6: Effect of N-Arachidonoyl Taurine on Cardiomyocytes

Cardiomyocytes were isolated from rat heart and the spontaneous electrical activity in the cardiomyocytes was measured using the current clamp mode of patch clamp. The application of 30 μM of N-AT reduced the length of the ventricular action potential (which corresponds to a reduction of the QT interval of the ECG) (FIG. 9A). This result demonstrates that N-AT can decrease the length of the ventricular action potential, which is what is needed to reverse the effect of Long QT mutations. Further, arrhythmia was induced in these cardiomyocytes by the I_{Ks} specific drug chromanol. In the presence of 5 μM chromanol, there are clearly additional action potentials caused by early after depolarizations (EADs) (FIG. 9D). EADs are known to accompany Long QT and could lead to ventricular fibrillation and cardiac sudden death in the intact heart. The application of 30 μMN-AT reversed the arrhythmic behavior of cardiomyocytes treated with chromanol (FIG. 9E). 30 μMN-AT (in the presence of 5 μM chromanol) removed these EADs (presumably by activating more I_{Ks} channels that keep the cells hyperpolarized at the end of the action potential and thereby preventing the EADs) and restored the rhythmic behavior of the cardiomyocytes to that before application of chromanol (compare FIGS. 9B and 9E). N-AT and chromanol are known to bind to different parts of the I_{Ks} channels (chromanol is a pore blocker, whereas N-AT is binding to the voltage sensor), so this effect of N-AT is not just a competition with chromanol to the same binding site. Rather, N-AT increases the currents by attracting the voltage sensor, whereas chromanol is known to block the currents by binding to the pore. These data demonstrate that N-AT has anti-arrhythmic activity in rat cardiomoycytes and suggests that N-AT can reverse I_{Ks}-induced arrhythmias, such as Long QT syndrome.

TABLE 1
Effect of PUFAs on Channel Voltage Dependence

| | Construct | Compound | pH | ΔV_{1/2} ± SEM (mV) | n | P† |
|---|---|---|---|---|---|---|
| [symbol] | Kv7.1 | 70 μM DHA | 7.4 | −9.3 ± 0.9 | 3 | 0.009 |
| | Kv7.1 | 70 μM DHA | 9.0 | −25.1 ± 4.2 | 5 | 0.004 |
| | Kv7.1 | 70 μM EPA | 7.4 | −12.7 ± 1.4 | 3 | 0.01 |
| | Kv7.1 | 70 μM OA | 7.4 | −1.0 ± 0.5 | 3 | 0.2 |
| | Kv7.1 | 70 μM DHA-me | 7.4 | +2.8 ± 2.5 | 3 | 0.4 |
| | Kv7.1 | 70 μM AA+ | 7.4 | +9.7 ± 2.1 | 8 | 0.002 |
| | Kv7.1 | 70 μM DHA-Gly | 7.4 | −24.3 ± 2.3 | 5 | 0.0005 |
| | Kv7.1 | 70 μM N—AT | 7.4 | −26.2 ± 2.7 | 4 | 0.003 |
| | Kv7.1/R228Q | 70μM DHA-Gly | 7.4 | −0.6 ± 1.2 | 4 | 0.9 |
| [symbol] | Kv7.1/E1 | 70 μM DHA | 7.4 | −0.2 ± 1.1 | 3 | 0.9 |
| | Kv7.1/E1 | 70 μM DHA | 9.0 | −25.7 ± 3.3 | 4 | 0.004 |
| | Kv7.1/E1 | 70 μM AA+ | 7.4 | +22.6 ± 1.9 | 10 | <0.0001 |
| | Kv7.1/E1 | 70 μM DHA-Gly | 7.4 | −25.1 ± 3.9 | 6 | 0.001 |
| | Kv7.1/E1 | 70 μM N—AT | 7.4 | −27.0 ± 2.5 | 5 | 0.0004 |
| [symbol] | Kv7.1/E1ΔC | 70 μM DHA | 7.4 | −1.0 ± 1.7 | 8 | 0.6 |
| [symbol] | E1-Kv7.1 | 70 μM DHA | 7.4 | +5.9 ± 1.4 | 15 | 0.0007 |
| | E1-Kv7.1 | 70 μM DHA | 9.0 | −27.9 ± 4.7 | 6 | 0.002 |
| | E1-Kv7.1 | 70 μM DHA-me | 7.4 | +5.0 ± 1.3 | 5 | 0.02 |
| | E1-Kv7.1 | 70 μM DHA-me | 9.0 | +3.6 ± 2.6 | 4 | 0.3 |
| [symbol] | E1-Kv7.1-Kv7.1 | 70 μM DHA | 7.4 | +5.0 ± 1.9 | 13 | 0.02 |
| | E1-Kv7.1-Kv7.1 | 70 μM DHA | 9.0 | −30.4 ± 2.5 | 6 | <0.0001 |
| | E1-Kv7.1-Kv7.1 | 70 μM DHA-me | 7.4 | +5.7 ± 0.9 | 6 | 0.002 |
| | E1-Kv7.1-Kv7.1 | 70 μM DHA-me | 9.0 | +8.2 ± 2.7 | 3 | 0.1 |

†from two-tailed one sample t-test where mean values were compared with 0.
DHA = docosahexaenoic acid,
EPA = eicosapentaenoic acid,
OA = oleic acid,
DHA-me = docosahexaenoic acid methyl ester,
AA++ = arachidonyl amine,
DHA-Gly = docosahexaenoyl glycine,
N—AT = N-arachidonoyl taurine.

TABLE 2
Summary of the effect of 30 μM N-AT on rat cardiomyocytes.

| | Control | N-AT | Change | | |
|---|---|---|---|---|---|
| | Mean ± SEM | Mean ± SEM | Mean ± SEM | P | n |
| APD30 | 261 ± 50 ms | 211 ± 37 ms | 0.81 ± 0.04* | 0.007† | 5 |
| APD50 | 341 ± 62 ms | 279 ± 47 ms | 0.83 ± 0.03* | 0.006† | 5 |
| APD70 | 402 ± 65 ms | 336 ± 50 ms | 0.84 ± 0.03* | 0.008† | 5 |
| RR-interval | 2869 ± 319 ms | 2075 ± 197 ms | 0.74 ± 0.05* | 0.009† | 5 |
| Amplₚₑₐₖ | 41 ± 3 mV | 32 ± 2 mV | −9 ± 1 mV | 0.003‡ | 5 |
| RMP | −58 ± 2 mV | −56 ± 2 mV | +2 ± 1 mV | 0.2‡ | 5 |

†from two-tailed one sample t-test where mean values were compared with 1.
‡from two-tailed paired t-test.
*relative change.
N-AT = N-arachidonoyl taurine, APD = action potential duration, RR-interval = the inverse of action potential frequency, Amplₚₑₐₖ = peak action potential amplitude, RMP = resting membrane potential.

### Example 7: Restoration of Drug-Induced Long QT Syndrome

The PUFAs disclosed herein were found to restore the QT interval in intact hearts that were subjected to drug-induced Long QT syndrome. Long QT syndrome was induced in intact guinea pig hearts by administration of the drug E4031, a HERG channel blocker. Administration of N-AT and DHA-Gly restored the QT interval in the intact hearts, as shown in FIGS. 10A, 10B, 10C, 10D, and 10E, with DHA-Gly able to restore the QT intervals at lower concentrations (e.g., 10 μM) than N-AT.

### Example 8: Effect of PUFA Structure on Kv7.1 Affinity

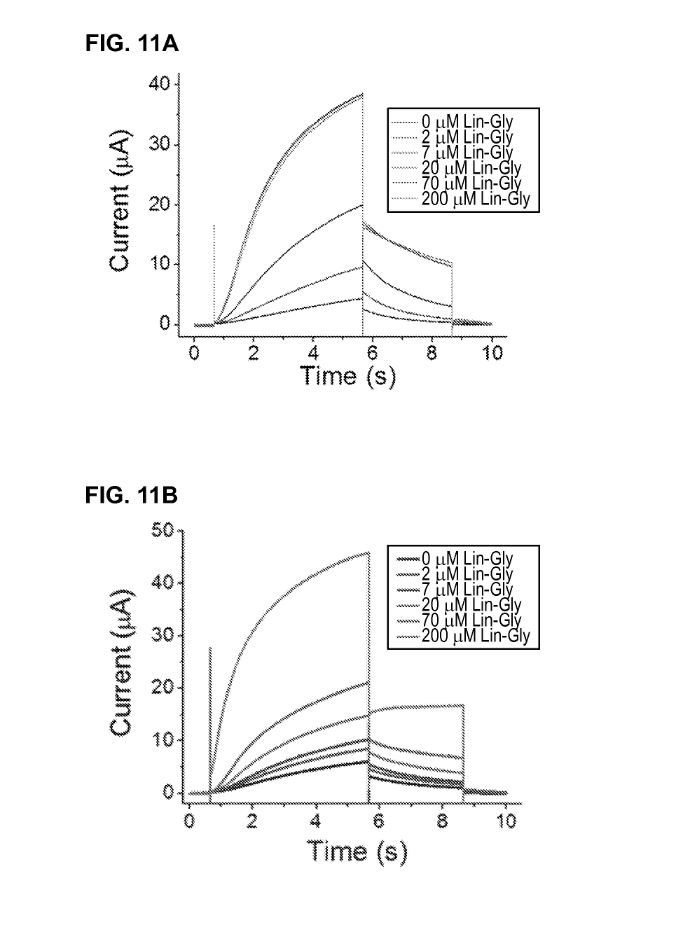

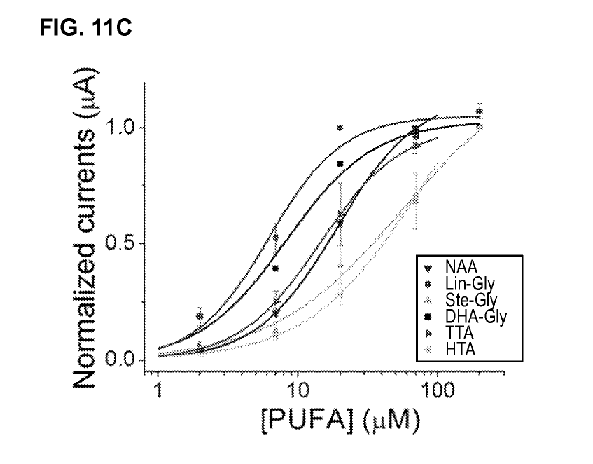

The effect of PUFA structure on Kv7.1 affinity was testing using NAA, Lin-Gly, Ste-GLy, DHA-Gly, TTA, and HTA, as previously described herein (FIG. 11). Of the PUFAs tested, Lin-Gly exhibited the lowest affinity.

### Example 9: In Vivo Permeability in Caco-2 Cells

The PUFAs disclosed herein were found to exhibit goodin vitro absorption into Caco-2 cells derived from human colon carcionoma, compared to reference compounds atenolol (paracellular transport) and propranolol (passive transcellular transport). In particular, DHA-gly had a permeability coefficient (Papp) value of 10×10⁶ cms⁻¹ and N-AT had a Papp value of 22×10⁻⁶ cms⁻¹. As a comparison, controls atenolol and propranolol, have Papp values of 0.6×10⁶ cms and 20×10⁻⁶ cms⁻¹, respectively. Atenolol has a known human absorption of 50% and propranolol 90%. Therefore, both N-AT and DHA-glycine should penetrate cells and be absorbed in in vivo. Further, DHA-gly has an efflux ratio of 3.6 and N-AT of 1.9. These values compare to atenolol, which has an efflux of 1, and propranolol, which has an efflux of 0.8.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

We claim:

1. A method of modulating a I_{Ks} channel comprising contacting the I_{Ks} channel with a compound, A-T, in an amount effective to modulate the I_{Ks} channel, wherein:
A is D(CH₂)ₘNHC(O), D(CHCH₃)ₘNHC(O), D(CH₂)ₘOC(O), D(CH₂)ₘSC(O), NH₂, NHR¹, N(R¹)₂, or N(R¹)₃⁺;
D is CO₂H, SO₃H, or OSO₃H;
T is C₅-C₂₉ alkenyl having at least two double bonds;
each R¹ independently is C₁₋₃ alkyl; and
m is 1-6.

2. The method of claim 1, wherein T is C₉₋₂₁ alkenyl.

3. The method of claim 2, wherein T is C₁₁₋₂₁ alkenyl.

4. The method of claim 3, wherein T is C₁₃₋₂₁ alkenyl.

5. The method of claim 4, wherein T is C₁₅₋₂₁ alkenyl.

6. The method of claim 5, wherein T is C₁₉₋₂₁ alkenyl.

7. The method of any one of claims 1-6, wherein T has at least three double bonds.

8. The method of claim 7, wherein T has at least four double bonds.

9. The method of claim 8, wherein T has at least five double bonds.

10. The method of claim 9, wherein T has at least six double bonds.

11. The method of any one of claims 1 to 10, wherein T has two to ten double bonds.

12. The method of any one of claims 1 to 11, wherein at least one double bond has cis stereochemistry.

13. The method of claim 12, wherein at least two double bonds have cis stereochemistry.

14. The method of any one of claims 1 to 13, wherein each of the double bounds have cis stereochemistry.

15. The method of any one of claims 1 to 14, wherein T is a linear alkenyl.

16. The method of any one of claims 1 to 15, wherein:
T comprises the structure: 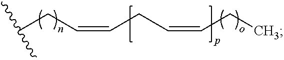
n is 1-6;
o is 0-6; and
p is 1-7.

17. The method of claim 16, wherein n is 1.

18. The method of claim 16, wherein n is 2.

19. The method of claim 16, wherein n is 3.

20. The method of claim 16, wherein n is 4.

21. The method of claim 16, wherein n is 5.

22. The method of claim 16, wherein n is 6.

23. The method of any one of claims 16 to 22, wherein o is 0.

24. The method of any one of claims 16 to 22, wherein o is 1.

25. The method of any one of claims 16 to 22, wherein o is 2.

26. The method of any one of claims 16 to 22, wherein o is 3.

27. The method of any one of claims 16 to 22, wherein o is 4.

28. The method of any one of claims 16 to 22, wherein o is 5.

29. The method of any one of claims 16 to 22, wherein o is 6.

30. The method of any one of claims 16 to 29, wherein p is 1.

31. The method of any one of claims 16 to 29, wherein p is 2.

32. The method of any one of claims 16 to 29, wherein p is 3.

33. The method of any one of claims 16 to 29, wherein p is 4.

34. The method of any one of claims 16 to 29, wherein p is 5.

35. The method of any one of claims 16 to 29, wherein p is 6.

36. The method of any one of claims 16 to 29, wherein p is 7.

37. The method of any one of claims 1 to 36, wherein A-T activates the I_{Ks} channel.

38. The method of claim 37, wherein A is D(CH₂)ₘNHC(O), D(CH₂)ₘOC(O), or D(CH₂)ₘSC(O).

39. The method of claim 38 wherein m is 1.

40. The method of claim 38, wherein m is 2.

41. The method of claim 38, wherein m is 3.

42. The method of claim 38, wherein m is 4.

43. The method of claim 38, wherein m is 5.

44. The method of claim 38, wherein m is 6.

45. The method of any one of claims 38 to 44, wherein A is D(CH₂)ₘNHC(O).

46. The method of claim 45, wherein D is COOH.

47. The method of claim 45, wherein D is SO₃H or OSO₃H.

48. The method of any one of claims 38 to 44, wherein A is D(CH₂)ₘOC(O).

49. The method of claim 48, wherein D is COOH.

50. The method of claim 48, wherein D is SO₃H or OSO₃H.

51. The method of any one of claims 38 to 44, wherein A is D(CH₂)ₘSC(O).

52. The method of claim 51, wherein D is COOH.

53. The method of claim 51, wherein D is SO₃H or OSO₃H.

54. The method of claim 38, wherein A-T is selected from the group consisting of:

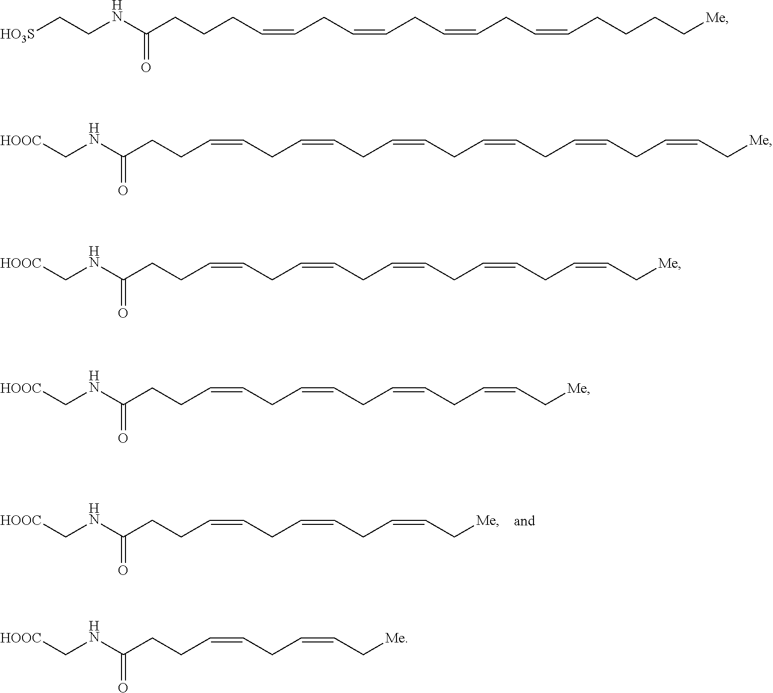 [SMILES: CCCCC/C=C\C/C=C\C/C=C\C/C=C\CCCC(=O)NCCS(=O)(=O)O ; CC/C=C\C/C=C\C/C=C\C/C=C\C/C=C\C/C=C\CCC(=O)NCC(=O)O ; CC/C=C\C/C=C\C/C=C\C/C=C\C/C=C\CCC(=O)NCC(=O)O ; CC/C=C\C/C=C\C/C=C\C/C=C\CCC(=O)NCC(=O)O ; CC/C=C\C/C=C\C/C=C\CCC(=O)NCC(=O)O ; CC/C=C\C/C=C\CCC(=O)NCC(=O)O]

55. The method of claim 38, wherein A-T is selected from the group consisting of:

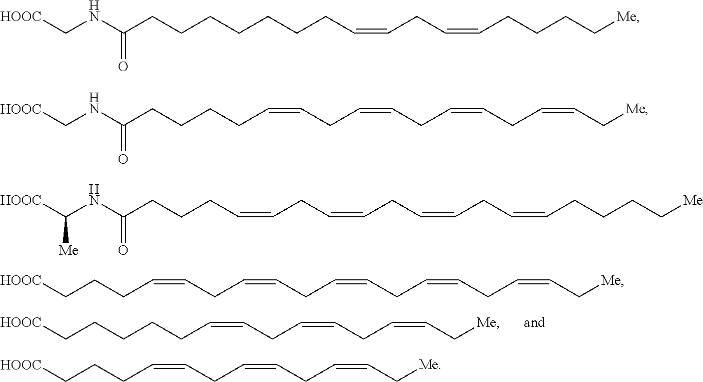

56. The method of any one of claims 1 to 37, wherein A-T inhibits the I_{Ks} channel.

57. The method of claim 56, wherein A is NH₂.

58. The method of claim 56, wherein A is NHR¹.

59. The method of claim 58, wherein R¹ is CH₃.

60. The method of claim 56, wherein A is N(R¹)₂.

61. The method of claim 60, wherein each k is CH₃.

62. The method of claim 56, wherein A is N(R¹)₃.

63. The method of claim 62, wherein each R¹ is CH₃.

64. The method of claim 56, wherein A-T is:

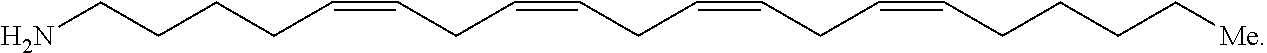 [SMILES: CCCCC/C=C\C/C=C\C/C=C\C/C=C\CCCCN]

65. The method of any one of claims 1 to 64, wherein A-T modulates the I_{Ks} channel under physiological conditions.

66. The method of any one of claims 1 to 65, wherein the contacting is in vivo.

67. The method of claim 66, wherein the contacting comprises administering A-T to a subject in need thereof.

68. The method of claim 67, wherein the subject suffers from Long QT Syndrome, cardiac arrhythmia, atrial flutter, ventricular tachycardia, Romano-Ward syndrome, Jervell and Lange-Nielsen syndrome, or drug-induced Long QT syndrome, and A-T activates the I_{Ks} channel.

69. The method of claim 67, wherein the subject suffers from Short QT Syndrome, blood clot formation, or atrial fibrillation and A-T inhibits the I_{Ks} channel.

70. The method of claim 68, wherein the Long QT Syndrome is drug-induced.

71. The method of any one of claims 1-68, wherein A-T is administered with a second therapeutic agent.

72. The method of claim 71, wherein the second therapeutic agent is an anti-cancer drug.
